(19) United States
(12) Patent Application Publication
(10) Pub. No.: US 2022/0401217 A1
(43) Pub. Date: Dec. 22, 2022

(54) AN IMPLANTABLE DEVICE FOR REPAIRING A CARDIAC VALVE

(71) Applicant: TAU CARDIO INC., Centreville, VA (US)

(72) Inventor: Jung-Hong Kim, Busan (KR)

(21) Appl. No.: 17/773,652

(22) PCT Filed: Nov. 4, 2020
(86) PCT No.: PCT/US2020/058787

§ 371 Date: May 2, 2022

Related U.S. Application Data
(60) Provisional application No. 63/109,322, filed on Nov. 3, 2020.
(60) Provisional application No. 62/930,599, filed on Nov. 5, 2019.

(51) Int. Cl. A61F 2/24

(52) U.S. Cl. CPC A61F 2/2445; A61B 2017/00243

(57) ABSTRACT

An implantable device is provided for repairing a cardiac valve having an annulus, two or more leaflets. The device comprises an annual member having a longitudinal axis and the annual member being dimensioned to attach to the annulus of the cardiac valve. The device further comprises a central member having a longitudinal axis, a distal portion, and a proximal portion. The central member is obliquely attached to the annual member in such a way the distal portion of the central member obliquely traverses the leaflets toward a ventricle to induce the leaflets to contact toward to the central member.

### RELATED APPLICATIONS

This application claims the benefit of U.S. priority of U.S. Provisional Patent Application No. 62/930,599 (filed on Nov. 5, 2019) and application No. 63/109,322 (filed on Nov. 3, 2020), the subject matter of which are hereby incorporated by reference in its entirety.

### TECHNICAL FIELD

This present invention generally relates to surgical apparatus and methods for treating dysfunctional heart valves, and more particularly to apparatus and related methods that provide valvular support to passively assist in preventing or mitigating heart valve regurgitation.

### BACKGROUND

Surgical correction of heart valve regurgitation is based upon overcorrection of concomitant annular dilatation using an undersized, complete, and rigid annuloplasty ring that is intended to reduce the diameter of the valve annulus and allow for leaflet coaptation. Although complete correction of the valve regurgitation has been surgically demonstrated, an important recurrence of the valve regurgitation after annuloplasty valve repair is common.

### SUMMARY

According to one embodiment, an implantable device is provided for repairing a cardiac valve having an annulus, two or more leaflets. The device comprises an annual member having a longitudinal axis and the annual member being dimensioned to attach to the annulus of the cardiac valve. The device further comprises a central member having a longitudinal axis, a distal portion, and a proximal portion. The central member is obliquely attached to the annual member in such a way the distal portion of the central member obliquely traverses the leaflets toward a ventricle to induce the leaflets to contact toward to the central member. The proximal portion of the central member is configured to be placed in an atrium. The device further comprises at least one connection member which is configured to support the central member from the annular member. The central member may have a shape of a croissant.

According to another embodiment, an implantable device is provided for repairing a cardiac valve having an annulus, two or more leaflets. The device comprises an annual member having a longitudinal axis in which the annual member is dimensioned to attach to the annulus of the cardiac valve. The device further comprises a central member having a longitudinal axis, a distal portion, and a proximal portion. The central member is obliquely attached to the annual member in such a way the longitudinal axes of the annual member and the central member have a predetermined angle to induce the leaflets to contact toward to the central member. The distal portion of the central member is configured to traverse the leaflets of the cardiac valve into a ventricle while the proximal portion of the central member is placed in an atrium. The entire central member may be configured to be placed in between the leaflets. The entire central member may be configured to be placed in an atrium. The entire central member may be configured to be placed in a ventricle. The predetermined angle may be about zero degree. The predetermined angle may be about 90 degree. The device further comprises at least one connection member configured to support the central member from the annular member. The central member may have a shape of a croissant.

According to one aspect of the present disclosure, a method is provided for treating regurgitation of blood flow through a diseased tricuspid valve. One step of the method includes providing an apparatus comprising an apparatus comprising a substantially annular support member and a central member securely connected thereto. Next, the substantially annular support member is attached to the annulus of the diseased tricuspid valve for the central member to obliquely extend behind the anterosuperior leaflet and extend in front of the posterior leaflet and the septal leaflet. According to another aspect of the present disclosure, a method is provided for treating regurgitation of blood flow through a diseased mitral valve. One step of the method includes providing an apparatus comprising an apparatus comprising a substantially annular support member and a central member securely connected thereto. Next, the substantially annular support member is attached to the annulus of the diseased mitral valve for the central member to obliquely extend between the anterior valve leaflet and the posterior valve leaflet.

### BRIEF DESCRIPTION OF THE DRAWINGS

FIG. 1A is a view showing a diseased tricuspid valve having regurgitation orifice (RO).

FIG. 1B is a view showing a diseased mitral valve having regurgitation orifice (RO).

FIG. 1C is a cross-sectional view showing of the regurgitation orifice (RO) and valve leaflets.

FIG. 2A is a front view showing the apparatus for a tricuspid valve.

FIG. 2B is a side view showing the apparatus in FIG. 2A.

FIG. 2C is a cross-sectional view showing the apparatus in FIG. 2A implanted on a tricuspid valve.

FIG. 2D is a front view showing an alternative configuration of FIG. 2A for a tricuspid valve.

FIG. 2E is a side view showing the apparatus in FIG. 2D.

FIG. 2F is a cross-sectional view showing the apparatus in FIG. 2D implanted on a tricuspid valve.

FIG. 3A is a front view showing an alternative configuration of the apparatus for a tricuspid valve.

FIG. 3B is a side view showing the apparatus in FIG. 3A.

FIG. 3C is a cross-sectional view showing the apparatus in FIG. 3A implanted on a tricuspid valve.

FIG. 3D is a front view showing an alternative configuration of FIG. 3A for a tricuspid valve.

FIG. 3E is a side view showing the apparatus in FIG. 3D.

FIG. 3F is a cross-sectional view showing the apparatus in FIG. 3D implanted on a tricuspid valve.

FIG. 4A is a front view showing an alternative configuration of the apparatus for a tricuspid valve.

FIG. 4B is a side view showing the apparatus in FIG. 4A.

FIG. 4C is a cross-sectional view showing the apparatus in FIG. 4A implanted on a tricuspid valve.

FIG. 4D is a front view showing an alternative configuration of FIG. 4A for a tricuspid valve.

FIG. 4E is a side view showing the apparatus in FIG. 4D.

FIG. 4F is a cross-sectional view showing the apparatus in FIG. 4D implanted on a tricuspid valve.

FIG. 5A is a perspective view showing the apparatus in FIG. 2A when the tricuspid valve is open.

FIG. 5B is a perspective view showing the apparatus in FIG. 2A when the tricuspid valve is closed.

FIG. 5C is a cross-sectional view showing the apparatus in FIG. 5B implanted on a tricuspid valve.

FIG. 5D is a perspective view showing the apparatus in FIG. 2D when the tricuspid valve is open.

FIG. 5E is a perspective view showing the apparatus in FIG. 2D when the tricuspid valve is closed.

FIG. 5F is a cross-sectional view showing the apparatus in FIG. 5D implanted on a tricuspid valve.

FIG. 6A is a front view showing an alternative configuration of the apparatus for a tricuspid valve.

FIG. 6B is a side view showing the apparatus in FIG. 6A.

FIG. 6C is a cross-sectional view showing the apparatus in FIG. 6A implanted on a tricuspid valve.

FIG. 6D is a front view showing an alternative configuration of FIG. 6A for a tricuspid valve.

FIG. 6E is a side view showing the apparatus in FIG. 6D.

FIG. 6F is a cross-sectional view showing the apparatus in FIG. 6D implanted on a tricuspid valve.

FIG. 7A is a front view showing the apparatus for a mitral valve.

FIG. 7B is a side view showing the apparatus in FIG. 7A.

FIG. 7C is a cross-sectional view showing the apparatus in FIG. 7A implanted on a mitral valve.

FIG. 7D is a front view showing an alternative configuration of FIG. 7A for a mitral valve.

FIG. 7E is a side view showing the apparatus in FIG. 7D.

FIG. 7F is a cross-sectional view showing the apparatus in FIG. 7D implanted on a mitral valve.

FIG. 8A is a front view showing an alternative configuration of the apparatus for a mitral valve.

FIG. 8B is a side view showing the apparatus in FIG. 8A.

FIG. 8C is a cross-sectional view showing the apparatus in FIG. 8A implanted on a mitral valve.

FIG. 8D is a front view showing an alternative configuration of FIG. 8A for a mitral valve.

FIG. 8E is a side view showing the apparatus in FIG. 8D.

FIG. 8F is a cross-sectional view showing the apparatus in FIG. 8D implanted on a mitral valve.

FIG. 9A is a front view showing an alternative configuration of the apparatus for a mitral valve.

FIG. 9B is a side view showing the apparatus in FIG. 9A.

FIG. 9C is a cross-sectional view showing the apparatus in FIG. 9A implanted on a mitral valve.

FIG. 9D is a front view showing an alternative configuration of FIG. 9A for a mitral valve.

FIG. 9E is a side view showing the apparatus in FIG. 9D.

FIG. 9F is a cross-sectional view showing the apparatus in FIG. 9D implanted on a mitral valve.

FIG. 10A is a front view showing an alternative configuration of the apparatus for a mitral valve.

FIG. 10B is a top view showing the apparatus in FIG. 10A.

FIG. 10C is a top view showing the apparatus in FIG. 10A implanted on a mitral valve.

FIG. 11A is a front view showing an alternative configuration of the apparatus for a mitral valve.

FIG. 11B is a side view showing the apparatus in FIG. 11A.

FIG. 11C is a cross-sectional view showing the apparatus in FIG. 11A implanted on a mitral valve.

FIG. 12A is a perspective view showing the apparatus implanted on the mitral valve.

FIG. 12B is another perspective view showing the apparatus implanted on the mitral valve.

FIG. 12C is a perspective view showing the apparatus of FIG. 12A.

FIG. 12D is a side view showing the apparatus in FIG. 12A.

FIG. 13A is a front view showing an alternative configuration of the apparatus for a mitral valve.

FIG. 13B is a side view showing the apparatus of FIG. 13A.

FIG. 13C is a cross-sectional view showing the apparatus of FIG. 13A implanted on a mitral valve.

FIG. 14A is a front view showing an alternative configuration of the apparatus for a mitral valve.

FIG. 14B is a side view showing the apparatus of FIG. 14A.

FIG. 14C is a cross-sectional view showing the apparatus in FIG. 14A implanted on a mitral valve.

FIG. 15A is a front view showing an alternative configuration of the apparatus for a mitral valve.

FIG. 15B is a side view showing the apparatus of FIG. 15A.

FIG. 15C is a cross-sectional view showing the apparatus of FIG. 15A implanted on a mitral valve.

FIG. 16A is a front view showing an alternative configuration of the apparatus for a mitral valve.

FIG. 16B is a side view showing the apparatus of FIG. 16A.

FIG. 16C is a cross-sectional view showing the apparatus of FIG. 16A implanted on a tricuspid valve.

FIG. 17A is a perspective view showing an alternative configuration of the apparatus for a tricuspid valve.

FIG. 17B is a side view showing the apparatus of FIG. 17A.

FIG. 17C is a cross-sectional view showing the apparatus of FIG. 17A implanted on a tricuspid valve.

FIG. 18A is a front view showing an alternative configuration of the apparatus for a tricuspid valve.

FIG. 18B is a side view showing the apparatus of FIG. 18A.

FIG. 18C is a cross-sectional view showing the apparatus of FIG. 18A implanted on a tricuspid valve.

FIG. 19A is a front view showing an alternative configuration of the apparatus for a tricuspid valve.

FIG. 19B is a side view showing the apparatus of FIG. 19A.

FIG. 19C is a cross-sectional view showing the apparatus in FIG. 19A implanted on a tricuspid valve.

FIG. 20A is a front view showing an alternative configuration of the apparatus for a tricuspid valve.

FIG. 20B is a side view showing the apparatus of FIG. 20A.

FIG. 20C is a cross-sectional top view showing the apparatus of FIG. 20A implanted on a tricuspid valve.

FIG. 21A is a front view showing an alternative configuration of the apparatus for a tricuspid valve.

FIG. 21B is a side view showing the apparatus of FIG. 21A.

FIG. 21C is a cross-sectional view showing the apparatus of FIG. 21A implanted on a tricuspid valve.

FIG. 22A is a front view showing an alternative configuration of the apparatus for a tricuspid valve.

FIG. 22B is a side view showing the apparatus of FIG. 22A.

FIG. 22C is a cross-sectional view showing the apparatus of FIG. 22A implanted on a tricuspid valve.

FIG. 23A is a front view showing an alternative configuration of the apparatus for a tricuspid valve.

FIG. 23B is a side view showing the apparatus of FIG. 23A.

FIG. 23C is a cross-sectional view showing the apparatus of FIG. 23A implanted on a mitral valve.

### DETAILED DESCRIPTION OF EXAMPLE EMBODIMENTS

To assist in understanding the invention, reference is made to the accompanying drawings to show by way of illustration specific embodiments in which the invention may be practiced. The drawings herein are not necessarily made to scale or actual proportions. For example, lengths and widths of the components may be adjusted to accommodate the page size.

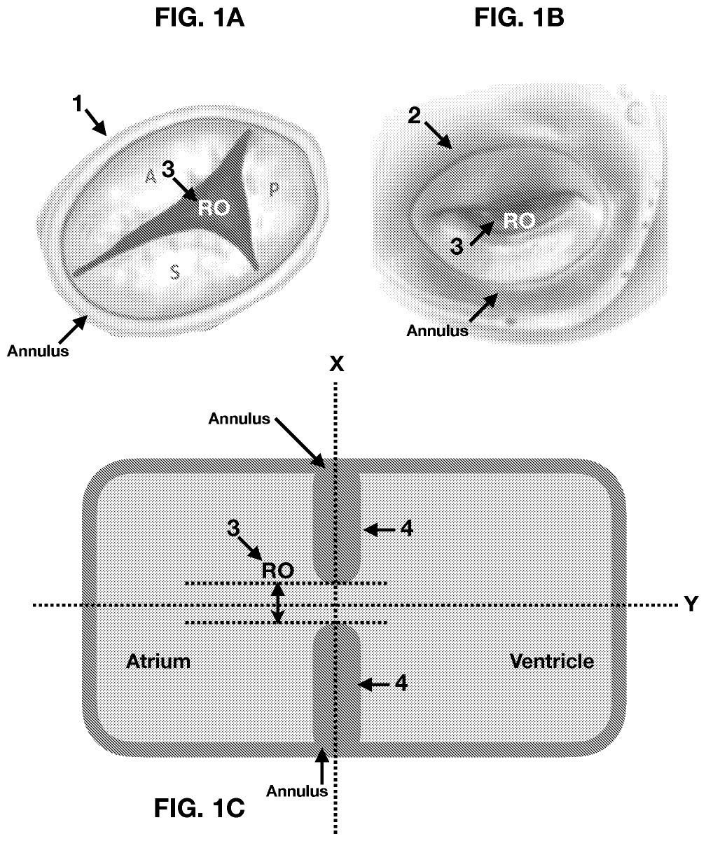

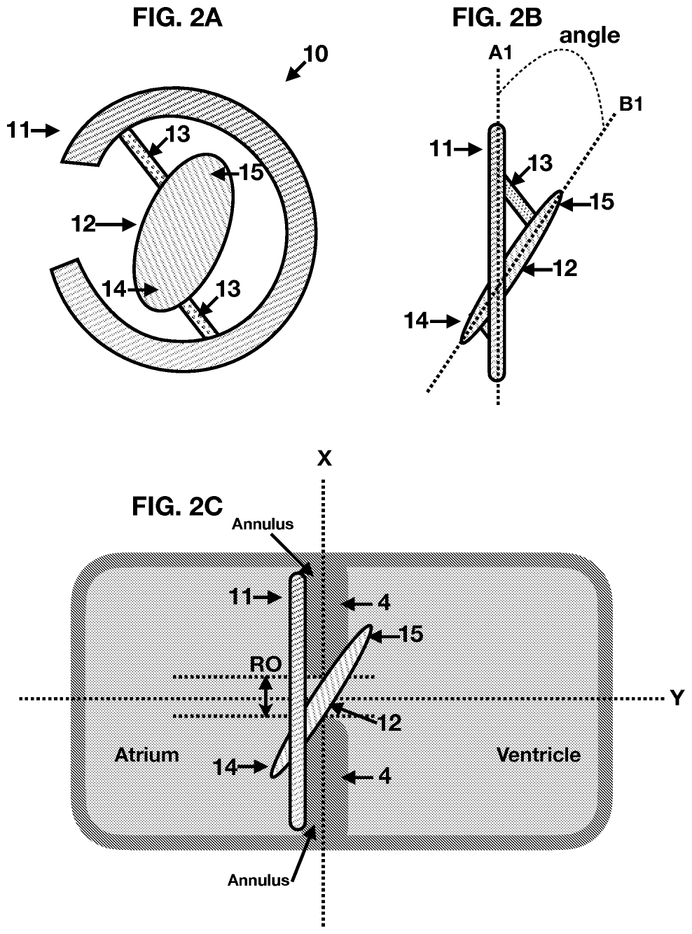

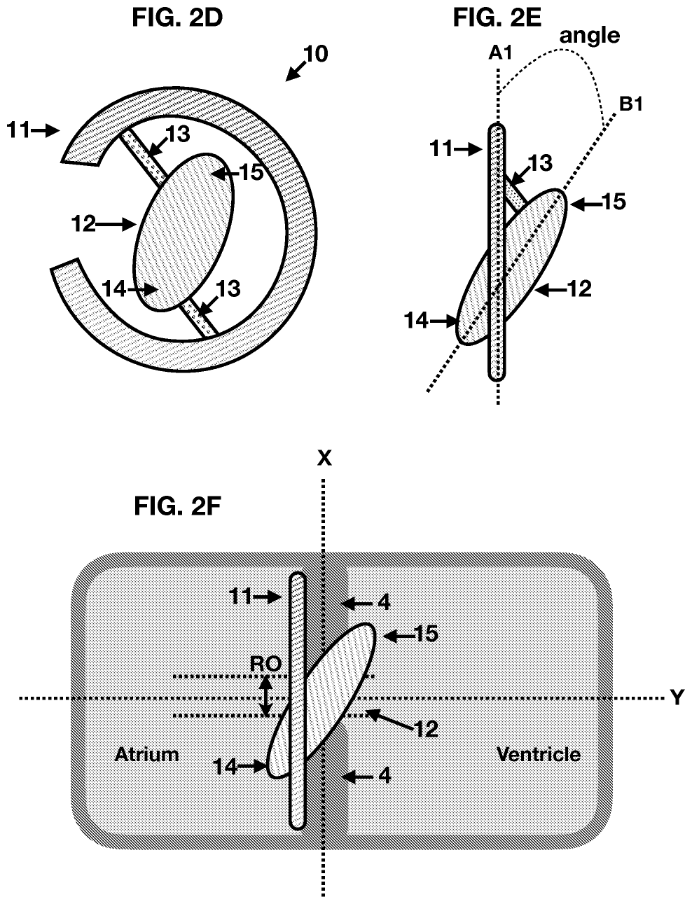

The present description relates to apparatus and methods for treating dysfunctional heart valves, and more particularly to apparatus and related methods that assist in preventing or mitigating heart valve regurgitation. As representative of one embodiment, FIGS. 2A-2C illustrate an apparatus 10 for treating regurgitation of blood flow through a regurgitation orifice (RO) 3, such as a tricuspid valve 1 and a mitral valve 2 as shown FIGS. 1A-B.

As shown in FIG. 1C, the diseased valve leaflets 4 with a RO 3 are illustrated in a cross-sectional view. The diseased valve leaflets 4 may include axis X and axis Y relative to the center of RO 3. As shown in FIG. 1C, the valve 4 may be located between the atrium and the ventricle, and functions to prevent the backflow of blood from the ventricle into the atrium during contraction.

Referring to FIGS. 2A-2C, the apparatus 10 for the tricuspid valve may comprise a substantially annular support member 11, a central member 12, and at least one connection member 13 securely connected between the annular support member 11 and the central member 12. As used herein, the term “substantially annular” can be used to describe an annular support member 11 having a circular or semi-circular configuration. Thus, the term “substantially annular” can refer to an annular support member 11 that is fully annular, fully circular, oval, partially circular, C-shaped (or reverse C-shape), D-shaped (or reverse D-shape), U-shaped (or reverse-U shape), etc.

As used herein, the term “substantially” can refer to the complete or nearly complete extent or degree of an action, characteristic, property, state, structure, item or result. For example, an annular support member 11 that is “substantially” annular mean that the support member 11 may be either completely annular or nearly completely annular. The exact allowable degree of deviation from absolute annularity may in some cases depend on the specific context. Generally speaking, however, the nearness of annularity will be so as to have the same overall results as if absolute and total annularity were obtained.

As shown in FIG. 2A, for example, the substantially annular support member 11 may be a reverse C-shaped and may be dimensioned for attachment to the annulus of the diseased tricuspid valve 1. As shown in FIG. 2B, the annular support member 11 may include a longitudinal axis A1. The substantially annular support 11 may have a rigid or semi-rigid configuration.

As FIG. 2A shows a front view of the apparatus 10, the apparatus 10 may also include at least one central member 12 that may be securely connected to the annular support member 11 by at least one connection member 13 at a first locations. As shown in FIGS. 2A-C, for example, the central member 12 may be connected to the annular support member 11 by at least two connection members 13. The central member 12 may include a longitudinal axis B1. The central member 12 may have a proximal portion 14, a distal portion 15, and a central portion thereof.

As FIG. 2B shows a top view of the apparatus 10, the central member 12 may be dimensioned, shaped, and configured to obliquely expand through the RO 3 when the apparatus 10 is implanted on or about the annulus of the diseased tricuspid valve 1 as shown in FIG. 2C. The oblique expansion may be defined as an angle created between the longitudinal axis A1 of the annular support member and the longitudinal axis B1 of the central member 12. The angle of the oblique expansion may be varying depending on patients.

As shown in FIG. 2C, the top view of the central member 12 may have a configuration where the central member 12 may obliquely expand through the RO 3 (i.e., between the diseased leaflets 4) with an angle between A1 and B1 as shown in FIG. 2B. The angle may be such that the central member 12 may induce the diseased leaflets 4 to contact toward to the central member 12. The angle of the oblique expansion may be varying depending on patients.

In one example of the embodiment, the angle may be between about 10 and about 60 (e.g., about 45 degree). To induce or attract the diseased leaflets 4 to contact toward to the central member 12, the connection member 13 may be selectively adjusted depending upon the anatomic leaflet configuration. It will be appreciated that the adjustment mechanism can be operated to adjust the lateral position of the central member 12 to adjust the angle defined between A1 and B1.

As FIG. 2C shows a top cross-sectional view of the apparatus 10 attached to the annulus of the diseased tricuspid valve 1, the central portion of the central member 12 may preferably be arranged at a position where axis X and axis Y may meet within the RO 3 for inducing free edge of the leaflets 4 to adhere toward the central member 12 during systole. As shown in FIG. 2C, the proximal portion 14 of the central member 12 may preferably be arranged to position in the atrium, and the distal portion 15 of the central member 12 may preferably be arranged to position within the ventricle. It should be appreciated that the such arrangement can be varied depending upon the diseased tricuspid valve anatomy.

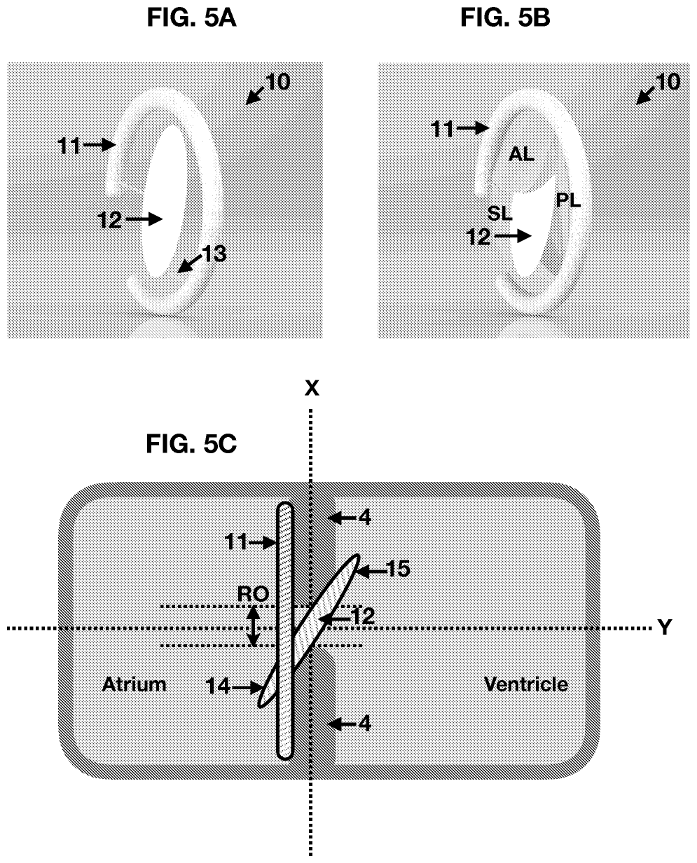

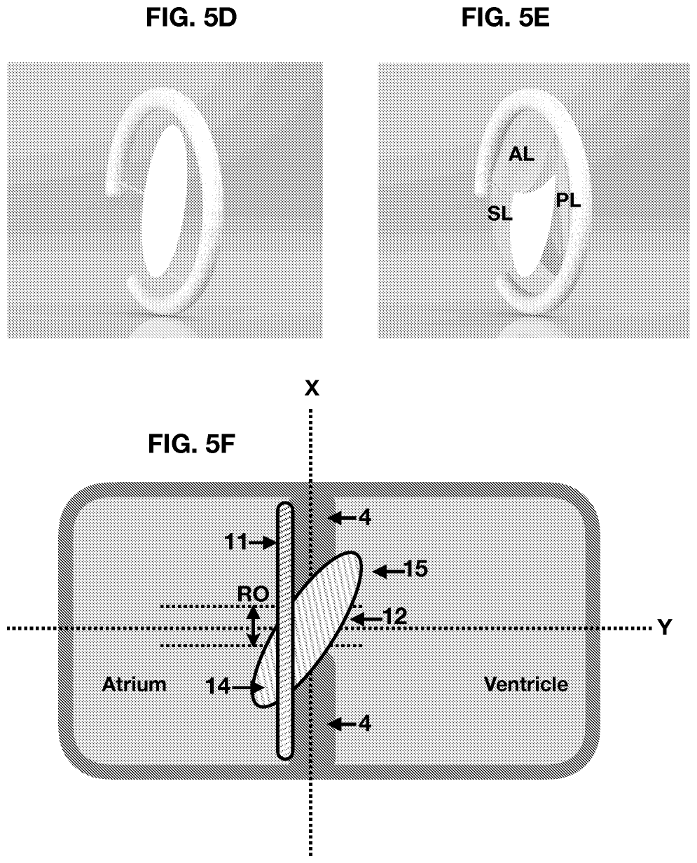

FIG. 5A is a perspective view of FIG. 2A. FIG. 5B shows that the diseased tricuspid valve leaflets 4 may be induced to adhere to the central member 12 when the diseased tricuspid valve is closed. As shown in FIGS. 5A-B, for example, the distal portion 15 of the central member 12 may preferably be configured to engage behind the anterosuperior leaflet 4, and the proximal portion 14 of the central member 12 may be configured to engage in front of the posterior leaflet and the septal leaflet during systole. It should be appreciated that the such engagement can be varied depending upon the diseased tricuspid valve anatomy.

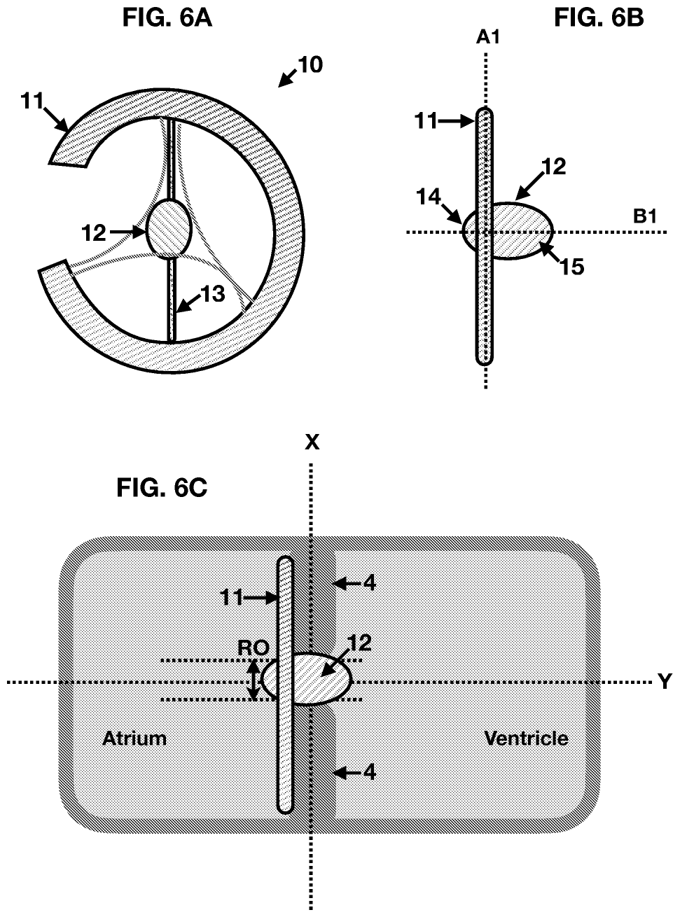

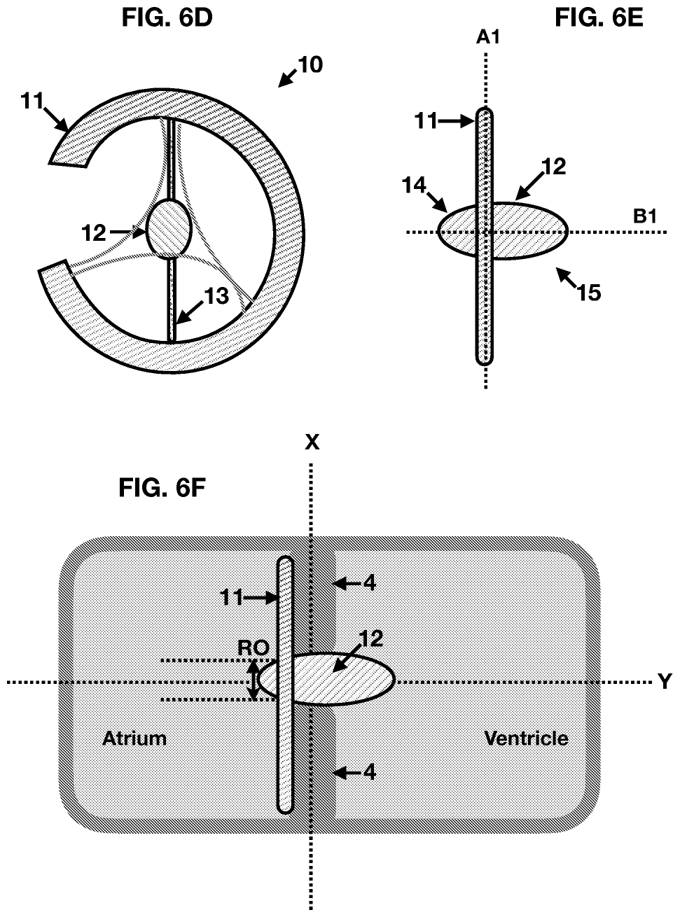

Referring back to FIGS. 2A-C, the central member 12 may have a rigid, semi-rigid, or flexible configuration. Where the central member 12 has a semi-rigid configuration, for example, the central member 12 may be bendable or adjustable to various positions. The central member 12 may have a 3D shape that may correspond to the actual 3D shape of the patient's valve leaflets 4. The engaging portion of the central member 12 may have an oval or elliptical shape. As shown in FIG. 6A-B, the central member 12 may have a convex shape relative to axis B1. It should be appreciated that the rigidity and shape of the central member 12 can be varied depending upon the diseased tricuspid valve anatomy for optimal leaflet coaptation. For example, FIGS. 2D-2F show the central member 12 may have a more convex or projecting shape than the one of FIGS. 2A-2C depending on patients.

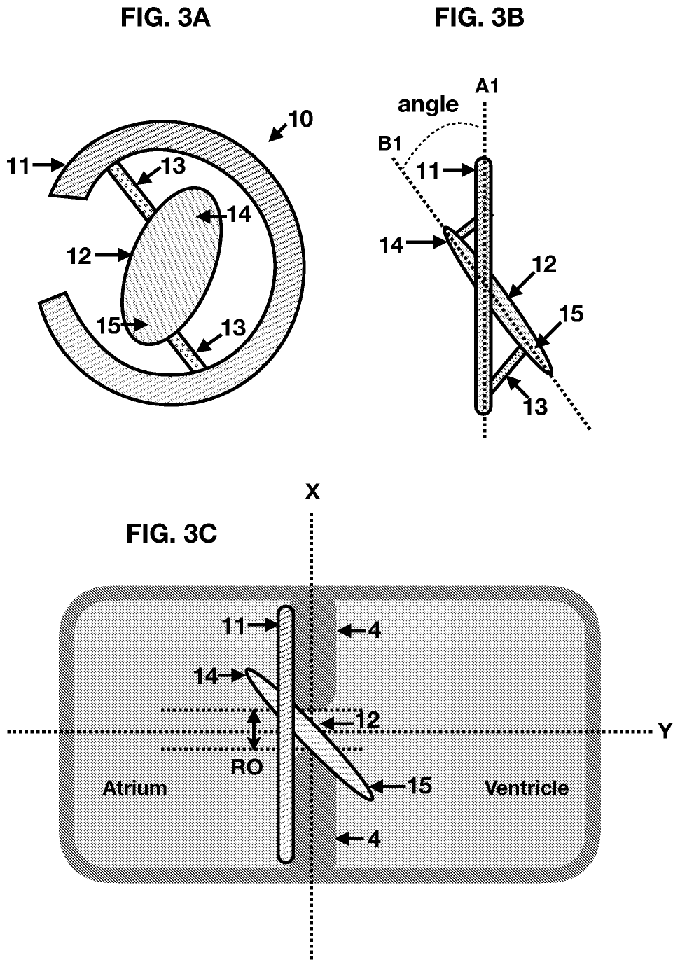

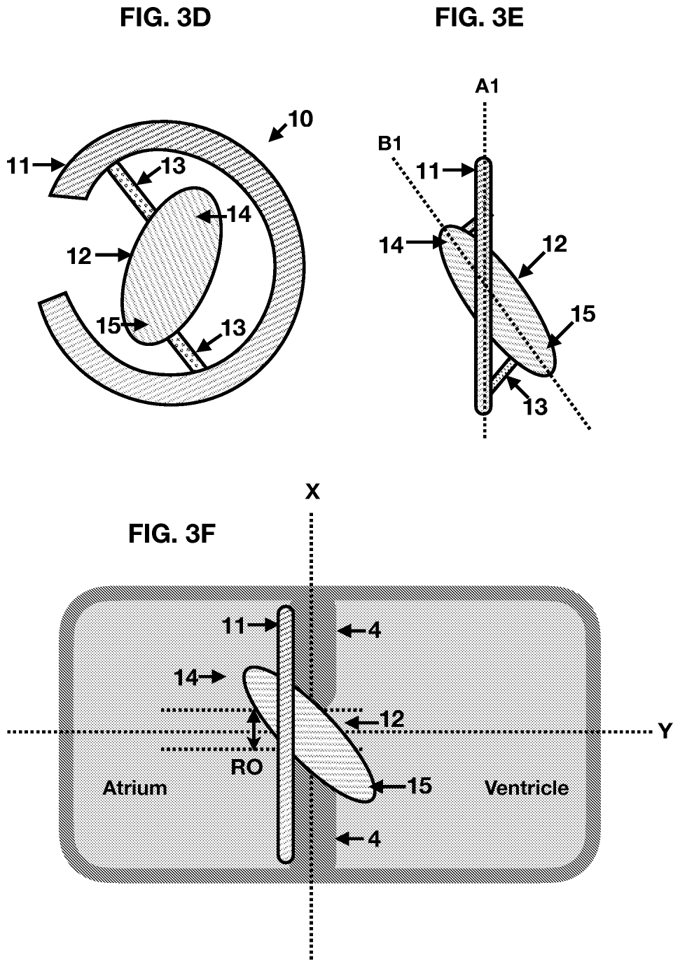

Another embodiment for the tricuspid valve is illustrated in FIGS. 3A-C. Referring to FIGS. 3A-C, the apparatus 10 may comprise a substantially annular support member 11, a central member 12, and at least one connection member 13 which may securely be connected between the annular support member 11 and the central member 12. As shown in FIG. 3A, for example, the substantially annular support member 11 may be a reverse C-shaped and may be dimensioned for attachment to the annulus of the diseased tricuspid valve 1. As shown in FIG. 3B, the annular support member 11 may include a longitudinal axis A1. The substantially annular support 11 may have a rigid semi-rigid, or flexible configuration.

As FIG. 3A shows a front view of the apparatus 10, the apparatus 10 may also include at least one central member 12 that may securely be connected to the annular support member 11 by at least one connection member 13 at a first locations. As shown in FIGS. 3A-C, for example, the central member 12 may be connected to the annular support member 11 by two connection members 13. The central member 12 includes a longitudinal axis B1. The central member 12 has a proximal portion 14, a distal portion 15, and a central portion.

As FIG. 3B shows a top view of the apparatus 10, the central member 12 may be dimensioned, shaped, and configured to obliquely expand downward through the RO 3 when the apparatus 10 is implanted on or about the diseased tricuspid valve 1 as shown in FIG. 3C. The oblique expansion may be defined as an angle created between the longitudinal axis A1 of the annular support member and the longitudinal axis B1 of the central member 12. The angle of the oblique expansion may be varying depending on patients.

As shown in FIG. 3C, the top view of the central member 12 has a configuration where the central member 12 may obliquely expand downward through the RO 3 (i.e., between the diseased leaflets 4) with an angle between A1 and B1 as shown in FIG. 3B. The angle is such that the central member 12 can induce the diseased leaflets 4 to contact toward to the central member 12. The angle of the oblique expansion may be varying depending on patients.

In one example of the embodiment, the angle of A1 and B1 can be between about 10 and about 60 (e.g., about 45 degree). To induce the diseased leaflet 4 to contact toward to the central member 12, the connection member 13 can be selectively adjusted depending upon the anatomic leaflet configuration. It will be appreciated that the adjustment mechanism can be operated to adjust the lateral position of the central member 12 to adjust the angle defined between A1 and B1.

As FIG. 3C shows a top cross-sectional view of the apparatus 10 attached to the annulus of the diseased tricuspid valve 1, the central portion of the central member 12 is preferably arranged at a position where axis X and axis Y meet within the RO 3 for inducing free edge of the leaflets 4 to adhere or contact toward the central member 12 during systole. As shown in FIG. 3C, the distal portion 15 of the central member 12 is preferably arranged to position within the ventricle, and the proximal portion 14 of the central member 12 is preferably arranged to position within the atrium. It should be appreciated that the such arrangement can be varied depending upon the diseased tricuspid valve anatomy.

Referring to FIG. 3A-C, the central member 12 can have a rigid, semi-rigid, or flexible configuration. Where the central member 12 has a semi-rigid configuration, for example, the central member 12 can be bendable or adjustable to various positions. The central member 12 can have a 3D shape that corresponds to the 3D shape of the valve leaflets 4. The engaging portion of the central member 12 has an oval shape. Alternatively, the central member 12 can have a convex shape relative to axis B1. It should be appreciated that the rigidity and shape of the central member 12 can be varied depending upon the diseased tricuspid valve anatomy for optimal leaflet coaptation. For example, FIGS. 3D-3F show the central member 12 may have a more convex or projecting shape than the one of FIGS. 3A-3C depending on patients.

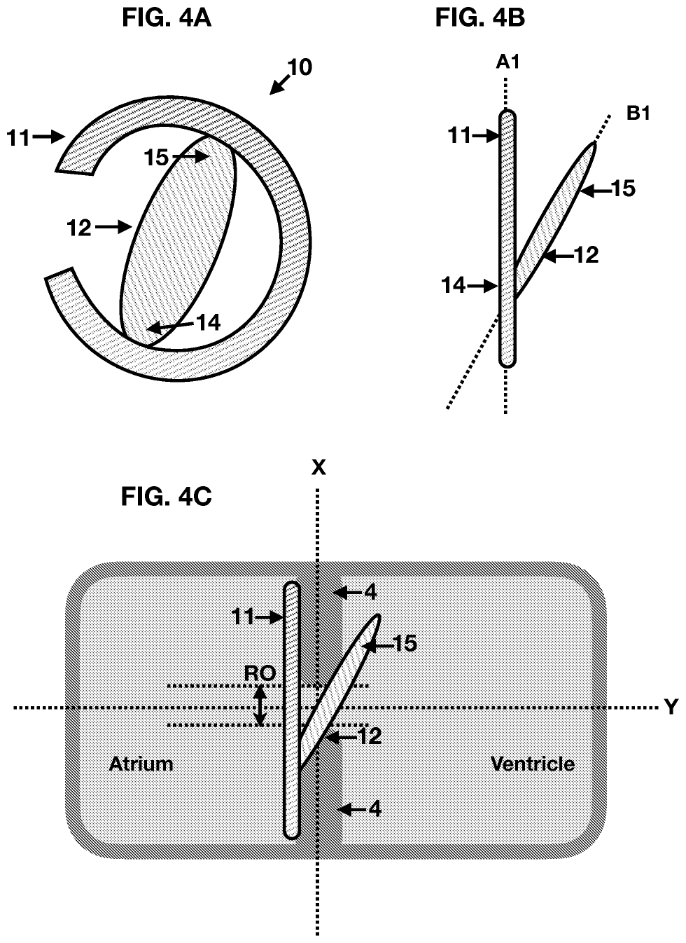

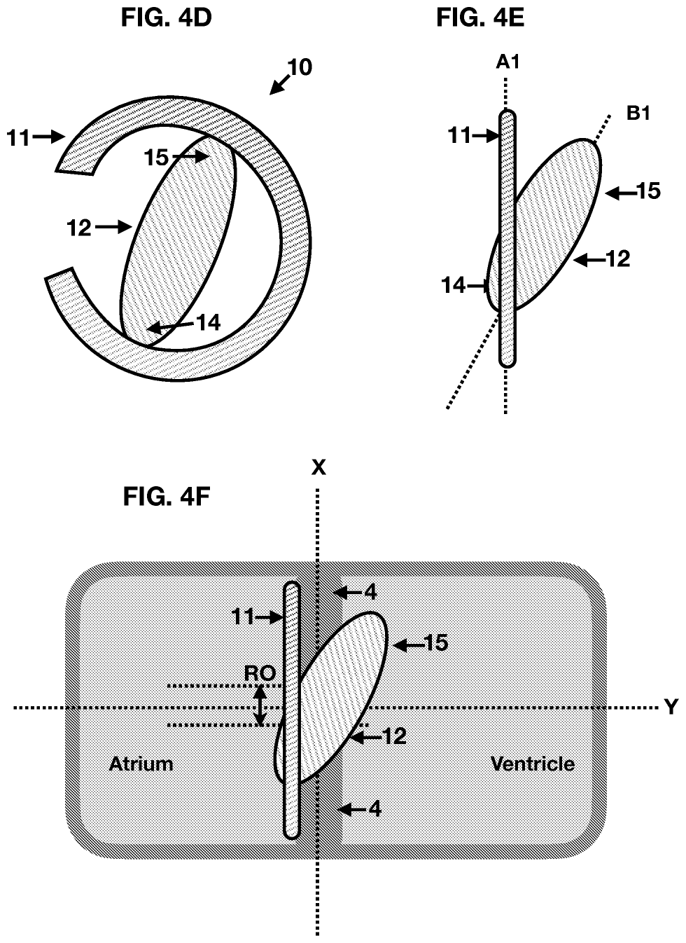

Another embodiment for the tricuspid valve is illustrated in FIGS. 4A-C. Referring to FIGS. 4A-C, the apparatus 10 comprises a substantially annular support member 11 and a central member 12 securely connected to the annular support member 11. As shown in FIG. 4A, for example, the substantially annular support member 11 can be a reverse C-shaped and is dimensioned for attachment to the annulus of the diseased tricuspid valve 1. As shown in FIG. 4B, the annular support member 11 includes a longitudinal axis A1. The central member 12 includes a longitudinal axis B1. The central member 12 has a proximal portion 14 and a distal portion 15.

As FIG. 4B shows a top view of the apparatus 10 of FIG. 4A, the central member 12 is dimensioned, shaped, and configured to obliquely expand upward through the RO 3 when the apparatus 10 is implanted on or about the diseased tricuspid valve 1 as shown in FIG. 4C. As shown in FIG. 4C, the central member 12 has a configuration where the central member 12 obliquely expands upward through the RO 3 (i.e., between the diseased leaflets 4) with an angle between A1 and B1 as shown in FIG. 4B. The angle is such that the central member 12 can induce the diseased leaflets 4 to contact toward to the central member 12. The oblique expansion may be defined as an angle created between the longitudinal axis A1 of the annular support member and the longitudinal axis B1 of the central member 12. The angle of the oblique expansion may be varying depending on patients.

In one example of the embodiment, the angle of A1 and B1 can be between about 10 and about 60 (e.g., about 45 degree). To induce the diseased leaflet 4 to contact toward to the central member 12, the dimension of the central member 12 can be selectively adjusted depending upon the anatomic leaflet configuration. It will be appreciated that the adjustment mechanism can be operated to adjust the lateral position of the central member 12 to adjust the angle defined between A1 and B1.

As FIG. 4C shows a top cross-sectional view of the apparatus 10 attached to the annulus of the diseased tricuspid valve 1, the central portion of the central member 12 is preferably arranged at a position where axis X and axis Y meet within the RO 3 for inducing free edge of the leaflets 4 to adhere or contact toward the central member 12 during systole. As shown in FIG. 4C, the proximal portion 14 of the central member 12 is preferably arranged to position within the atrium, and the distal portion 15 of the central member 12 is preferably arranged to position within the ventricle. It should be appreciated that the such arrangement can be varied depending upon the diseased tricuspid valve anatomy.

Referring to FIG. 4A-C, the central member 12 can have a rigid, semi-rigid, or flexible configuration. Where the central member 12 has a semi-rigid configuration, for example, the central member 12 can be bendable or adjustable to various positions. The central member 12 can have a 3D shape that corresponds to the 3D shape of the valve leaflets 4. The engaging portion of the central member 12 has an oval shape. Alternatively, the central member 12 can have a convex shape relative to axis B1. It should be appreciated that the rigidity and shape of the central member 12 can be varied depending upon the diseased tricuspid valve anatomy for optimal leaflet coaptation. For example, FIGS. 4D-4F show the central member 12 may have a more convex or projecting shape than the one of FIGS. 4A-4C depending on patients.

Another embodiment for the tricuspid valve is illustrated in FIG. 6A-C. Referring to FIGS. 6A-C, the apparatus 10 may comprise a substantially annular support member 11, a central member 12, and at least one connection member 13 which is securely connected between the annular support member 11 and the central member 12. As shown in FIG. 6A, for example, the substantially annular support member 11 can be a reverse C-shaped and is dimensioned for attachment to the annulus of the diseased tricuspid valve 1. As shown in FIG. 6B, the annular support member 11 includes a longitudinal axis A1. The substantially annular support 11 can have a rigid or semi-rigid configuration.

As FIG. 6A shows a front view of the apparatus 10, the apparatus 10 also includes at least one central member 12 that is securely connected to the annular support member 11 at a first locations via connection member 13. The central member 12 includes a longitudinal axis B1. The central member 12 has a proximal portion 14 and a distal portion 15. As FIG. 6B shows a top view of the apparatus 10 of FIG. 6A, the central member 12 is dimensioned, shaped, and configured to expand horizontally through the RO 3 when the apparatus 10 is implanted on or about the diseased tricuspid valve 1 as shown in FIG. 6C.

As shown in FIG. 6C, the top view of the central member 12 of FIG. 6A has a configuration where the central member 12 expands horizontally through the RO 3 (i.e., between the diseased leaflets 4) with an angle between A1 and B1 as shown in FIG. 4B. The angle is such that the central member 12 can induce the diseased leaflets 4 to contact toward to the central member 12. In one example of the embodiment, the angle of A1 and B1 can be about 90 degree. To induce the diseased leaflet 4 to contact toward to the central member 12, the dimension of the central member 12 can be selectively adjusted depending upon the anatomic leaflet configuration. It will be appreciated that the adjustment mechanism can be operated to adjust the lateral position of the central member 12 to adjust the angle defined between A1 and B1.

As FIG. 6C shows a top cross-sectional view of the apparatus 10 attached to the annulus of the diseased tricuspid valve 1, the central portion of the central member 12 is preferably arranged at a position where axis X and axis Y meet within the RO 3 for inducing free edge of the leaflets 4 to adhere or contact toward the central member 12 during systole. As shown in FIG. 6C, the proximal portion 14 of the central member 12 is preferably arranged to position within the atrium, and the distal portion 16 of the central member 12 is preferably arranged to position within the ventricle. It should be appreciated that the such arrangement can be varied depending upon the diseased tricuspid valve anatomy.

Referring to FIG. 6A-C, the central member 12 may have a rigid, semi-rigid, or flexible configuration. Where the central member 12 has a semi-rigid configuration, for example, the central member 12 can be bendable or adjustable to various positions. The central member 12 may have a 3D shape that may correspond to the 3D shape of the valve leaflets 4. The engaging portion of the central member 12 has an oval or elliptical shape. Alternatively, the central member 12 can have a convex shape relative to axis B1. It should be appreciated that the rigidity and shape of the central member 12 can be varied depending upon the diseased tricuspid valve anatomy for an optimal leaflet coaptation. For example, FIGS. 6D-6F show the central member 12 may have a more convex or projecting shape than the one of FIGS. 6A-6C depending on patients. For another example, the central member 12 may be shape like a croissant.

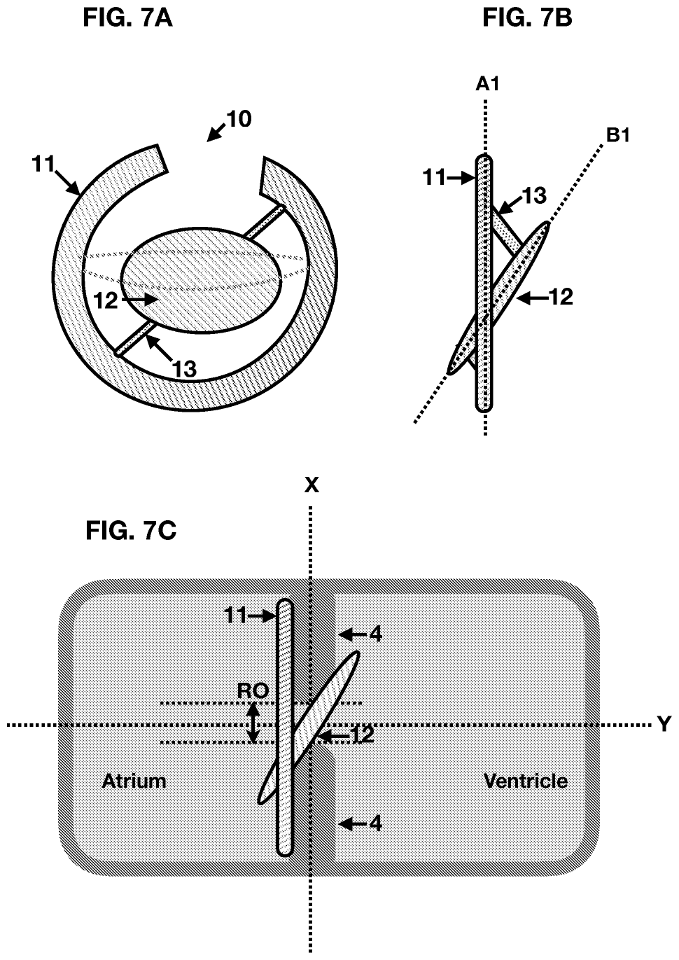

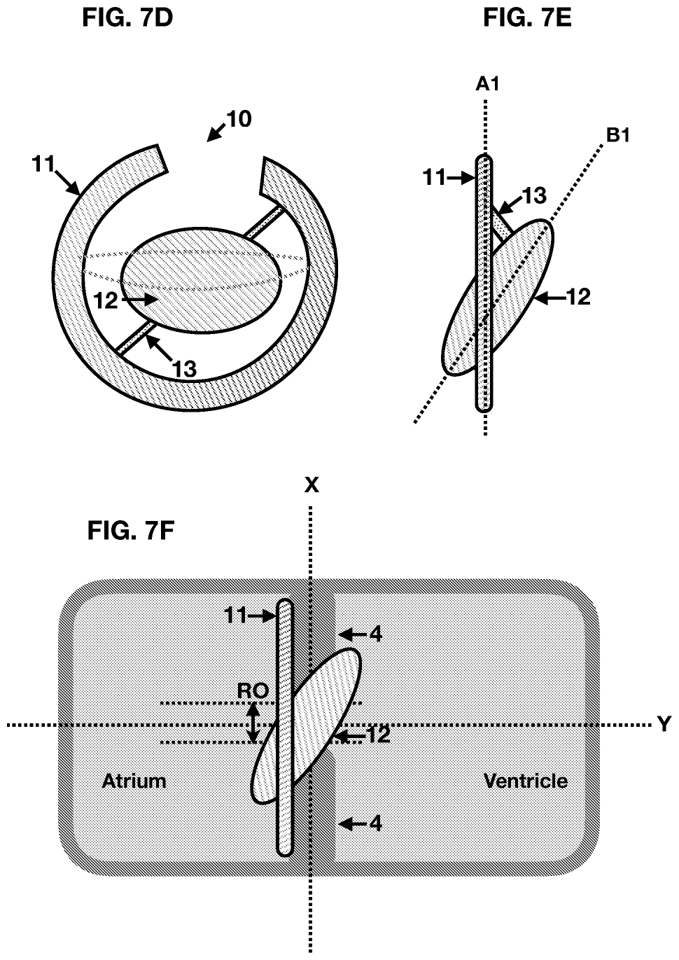

An embodiment for the mitral valve is illustrated in FIGS. 7A-C. Referring to FIGS. 7A-C, the apparatus 10 comprises a substantially annular support member 11, a central member 12, and at least one connection member 13 securely connected between the annular support member 11 and the central member 12. As shown in FIG. 7A, for example, the substantially annular support member 11 may be a U-shaped and is dimensioned for attachment to the annulus of the diseased mitral valve 2 as shown in FIG. 1B. As shown in FIG. 7B, the annular support member 11 may include a longitudinal axis A1. The substantially annular support 11 may have a rigid, semi-rigid, or flexible configuration.

As FIG. 7A shows a front view of the apparatus 10, the apparatus 10 may also include at least one central member 12 that is securely connected to the annular support member 11 at a first location via connection members 13. The central member 12 includes a longitudinal axis B1. The central member 12 has a proximal portion 14 and a distal portion 15. As FIG. 7B shows a top view of the apparatus 10 of FIG. 7A, the central member 12 is dimensioned, shaped, and configured to expand upward through the RO 3 when the apparatus 10 is implanted on or about the diseased mitral valve 2 as shown in FIG. 7C. For example, as shown in FIG. 7C, the central member 12 has a configuration where the central member 12 expands upward through the RO 3 (i.e., between the diseased leaflets 4) with an angle between A1 and B1 as shown in FIG. 7B. The angle is such that the central member 12 can induce the diseased leaflets 4 to contact toward the central member 12.

In one example of the embodiment, the angle of A1 and B1 can be about 10 and about 60 (e.g., about 45 degree). To induce the diseased leaflet 4 to contact toward to the central member 12, the dimension of the central member 12 can be selectively adjusted depending upon the anatomic leaflet configuration. It will be appreciated that the adjustment mechanism can be operated to adjust the lateral position of the central member 12 to adjust the angle defined between A1 and B1.

As FIG. 7C shows a top cross-sectional view of the apparatus 10 attached to the annulus of the diseased tricuspid valve 1, the central portion of the central member 12 is preferably arranged at a position where axis X and axis Y meet within the RO 3 for inducing free edge of the leaflets 4 to adhere or contact toward the central member 12 during systole. When a proximal portion of the central member 12 is arranged within the atrium, during systole the pressure between the portion and the adjacent leaflets is increased. At the same time, the distal portion of the central member 12 is arranged within the ventricle, during systole the pressure between the portion and the adjacent leaflets is decreased. The above interactions attract and enhance the coaptation between the diseased leaflets and the central member 12 during systole.

For example, as shown in FIG. 7C, the proximal portion of the central member 12 is arranged within the atrium for inducing the adjacent free edge of the diseased leaflets to adhere toward the central member 12 during systole. At the same time, the distal portion of the central member 12 is arranged within the ventricle for inducing the adjacent free edge of the diseased leaflets to adhere toward the central member 12 during systole. It should be appreciated that the such arrangement can be varied depending upon the diseased mitral valve anatomy.

Referring to FIG. 7A-7C, the central member 12 can have a rigid or semi-rigid configuration. Where the central member 12 has a semi-rigid configuration, for example, the central member 12 can be bendable or adjustable to various positions. The central member 12 can have a 3D shape that corresponds to the 3D shape of the valve leaflets 4. The engaging portion of the central member 12 has a oval shape. Alternatively, the central member 12 can have a convex shape relative to axis B1. It should be appreciated that the rigidity and shape of the central member 12 can be varied depending upon the diseased mitral valve anatomy for optimal leaflet coaptation. For example, FIGS. 7D-7F show the central member 12 may have a more convex or projecting shape than the one of FIGS. 7A-7C depending on patients.

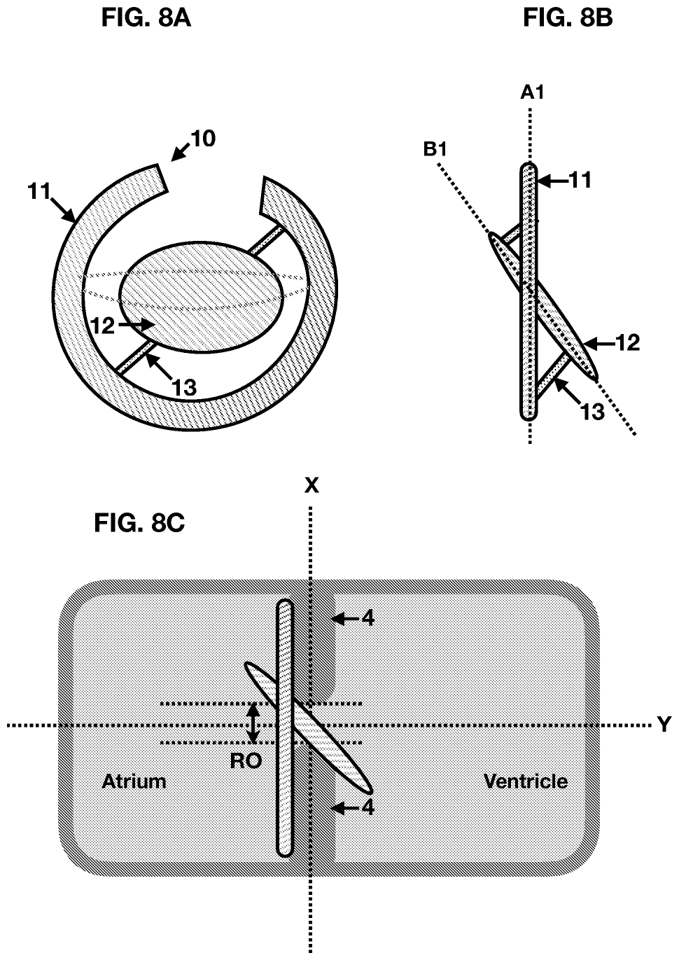

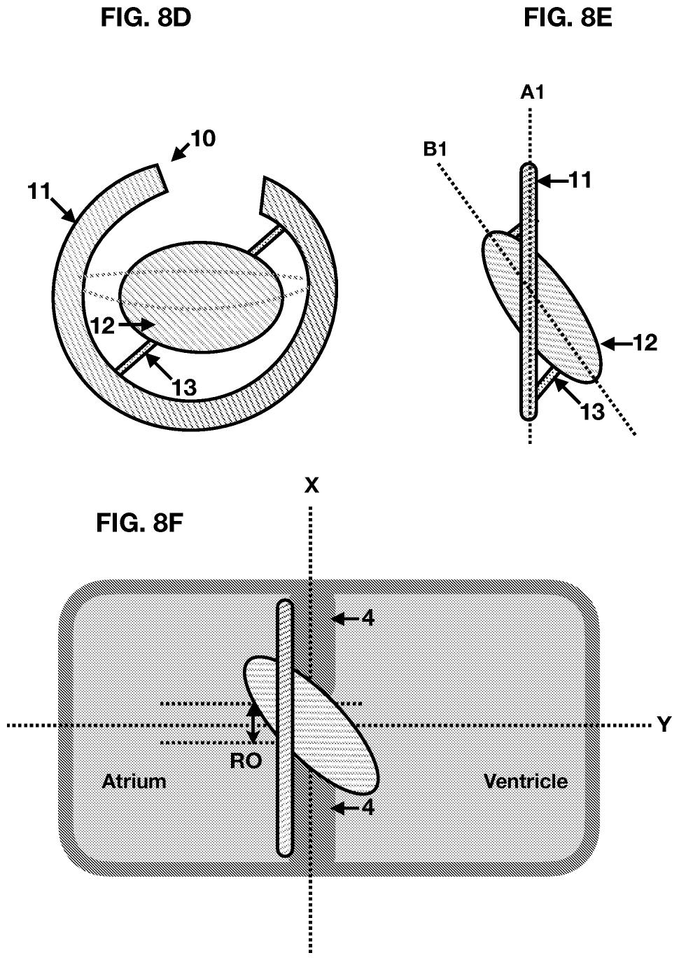

Another embodiment for the mitral valve is illustrated in FIGS. 8A-C. Referring to FIGS. 8A-C, the apparatus 10 comprises a substantially annular support member 11, a central member 12, and at least one connection member 13 securely connected between the annular support member 11 and the central member 12. As shown in FIG. 8A, for example, the substantially annular support member 11 can be a U-shaped and is dimensioned for attachment to the annulus of the diseased mitral valve 2 as shown in FIG. 1B. As shown in FIG. 8B, the annular support member 11 includes a longitudinal axis A1. The substantially annular support 11 can have a rigid or semi-rigid configuration.

As FIG. 8A shows a front view of the apparatus 10, the apparatus 10 also includes at least one central member 12 that is securely connected to the annular support member 11 at a first location via connection members 13. The central member 12 includes a longitudinal axis B1. The central member 12 has a proximal portion 14 and a distal portion 15.

As FIG. 8B shows a top view of the apparatus 10 of FIG. 8A, the central member 12 is dimensioned, shaped, and configured to expand downward through the RO 3 when the apparatus 10 is implanted on or about the diseased mitral valve 2 as shown in FIG. 8C. As shown in FIG. 8C, the central member 12 has a configuration where the central member 12 expands downward through the RO 3 (i.e., between the diseased leaflets 4) with an angle between A1 and B1 as shown in FIG. 8B. The angle is such that the central member 12 can induce the diseased leaflets 4 to contact toward to the central member 12.

In one example of the embodiment, the angle of A1 and B1 can be about 10 and about 60 (e.g., about 45 degree). To induce the diseased leaflet 4 to contact toward to the central member 12, the dimension of the central member 12 can be selectively adjusted depending upon the anatomic leaflet configuration. It will be appreciated that the adjustment mechanism can be operated to adjust the lateral position of the central member 12 to adjust the angle defined between A1 and B1.

As FIG. 8C shows a top cross-sectional view of the apparatus 10 attached to the annulus of the diseased tricuspid valve 1, the central portion of the central member 12 is preferably arranged at a position where axis X and axis Y meet within the RO 3 for inducing free edge of the leaflets 4 to adhere (or contact) toward the central member 12 during systole. When a proximal portion of the central member 12 is arranged within the atrium, during systole the pressure between the portion and the adjacent leaflets is increased. At the same time, a distal portion of the central member 12 is arranged within the ventricle, during systole the pressure between the portion and the adjacent leaflets is decreased. The above interactions attract and enhance the coaptation between the diseased leaflets and the central member 12 during systole. It should be appreciated that the such arrangement can be varied depending upon the diseased mitral valve anatomy.

Referring to FIG. 8A-C, the central member 12 can have a rigid or semi-rigid configuration. Where the central member 12 has a semi-rigid configuration, for example, the central member 12 can be bendable or adjustable to various positions. The central member 12 can have a 3D shape that corresponds to the 3D shape of the valve leaflets 4. The engaging portion of the central member 12 has an oval shape. Alternatively, the central member 12 can have a convex shape relative to axis B1. It should be appreciated that the rigidity and shape of the central member 12 can be varied depending upon the diseased mitral valve anatomy for optimal leaflet coaptation. For example, FIGS. 8D-8F show the central member 12 may have a more convex or projecting shape than the one of FIGS. 8A-8C depending on patients.

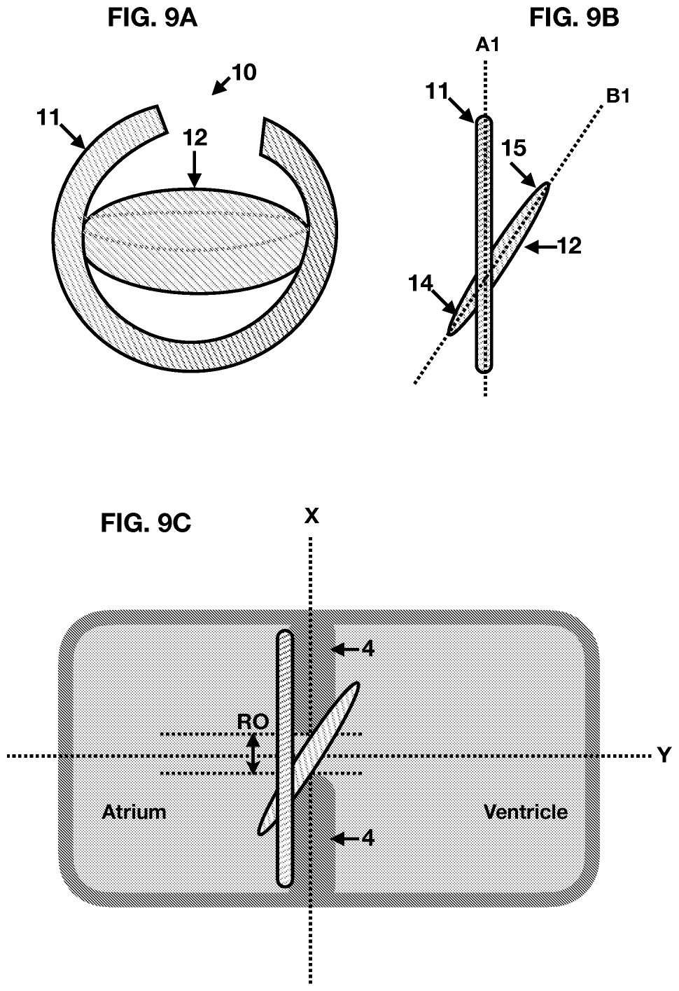

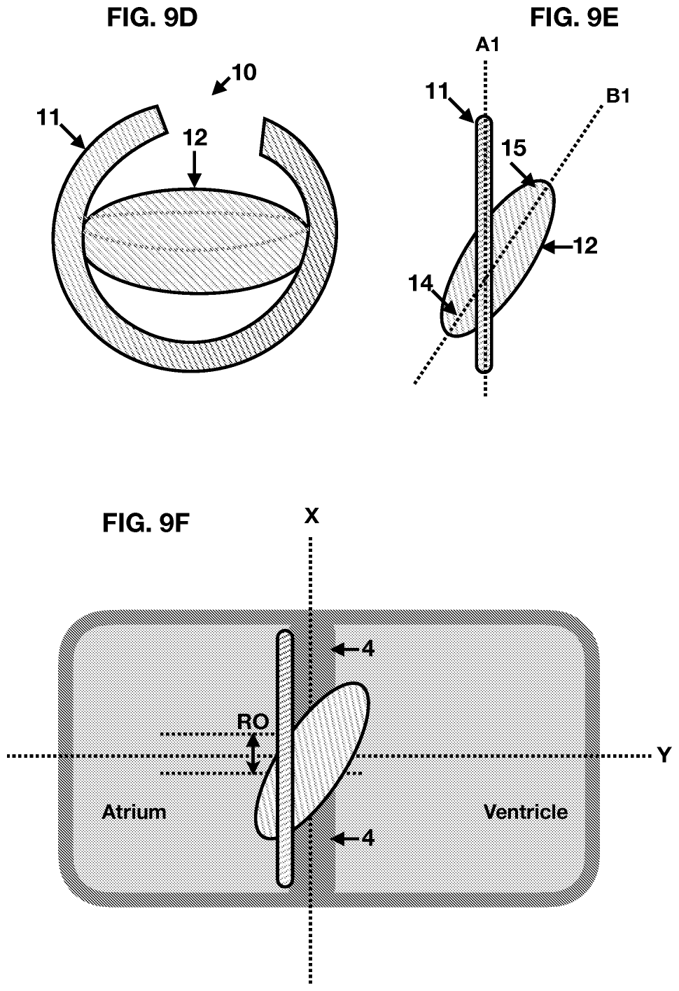

Another embodiment for the mitral valve is illustrated in FIGS. 9A-C. Referring to FIGS. 9A-C, the apparatus 10 comprises a substantially annular support member 11 and a central member 12 securely connected to the annular support member 11. As shown in FIG. 9A, for example, the substantially annular support member 11 can be a U-shaped and is dimensioned for attachment to the annulus of the diseased mitral valve 2 as shown in FIG. 1B. As shown in FIG. 9B, the annular support member 11 includes a longitudinal axis A1. The substantially annular support 11 can have a rigid, semi-rigid, or flexible configuration.

As shown in FIG. 9B, the central member 12 includes a longitudinal axis B1. The central member 12 has a proximal portion 14 and a distal portion 15. As FIG. 9B shows a top view of the apparatus 10 of FIG. 9A, the central member 12 is dimensioned, shaped, and configured to expand upward through the RO 3 when the apparatus 10 is implanted on or about the diseased mitral valve 2 as shown in FIG. 9C.

As shown in FIG. 9C, the central member 12 has a configuration where the central member 12 expands upward through the RO 3 (i.e., between the diseased leaflets 4) with an angle between A1 and B1 as shown in FIG. 9B. The angle is such that the central member 12 can induce the diseased leaflets 4 to contact toward to the central member 12. In one example of the embodiment, the angle of A1 and B1 can be about 10 and about 60 (e.g., about 45 degree). To induce the diseased leaflet 4 to contact toward to the central member 12, the dimension of the central member 12 can be selectively adjusted depending upon the anatomic leaflet configuration. It will be appreciated that the adjustment mechanism can be operated to adjust the lateral position of the central member 12 to adjust the angle defined between A1 and B1.

As FIG. 9C shows a top view of cross-sectional view of the apparatus 10 attached to the annulus of the diseased mitral valve 1, the central portion of the central member 12 is preferably arranged at a position where axis X and axis Y meet within the RO 3 for inducing free edge of the leaflets 4 to adhere or contact toward the central member 12 during systole. When a proximal portion 14 of the central member 12 is arranged within the atrium, during systole the pressure between the portion and the adjacent leaflets is increased. At the same time, when a distal portion 15 of the central member 12 is arranged within the ventricle during systole, the pressure between the portion and the adjacent leaflets is decreased. The above interactions attract and enhance the coaptation between the diseased leaflets and the central member 12 during systole. It should be appreciated that the such arrangement can be varied depending upon the diseased mitral valve anatomy.

Referring to FIG. 9A-C, the central member 12 can have a rigid or semi-rigid configuration. Where the central member 12 has a semi-rigid configuration, for example, the central member 12 can be bendable or adjustable to various positions. The central member 12 can have a 3D shape that corresponds to the 3D shape of the valve leaflets 4. The engaging portion of the central member 12 has a oval shape. Alternatively, the central member 12 can have a convex shape relative to axis B1. It should be appreciated that the rigidity and shape of the central member 12 can be varied depending upon the diseased mitral valve anatomy for optimal leaflet coaptation. For example, FIGS. 9D-9F show the central member 12 may have a more convex or projecting shape than the one of FIGS. 9A-9C depending on patients.

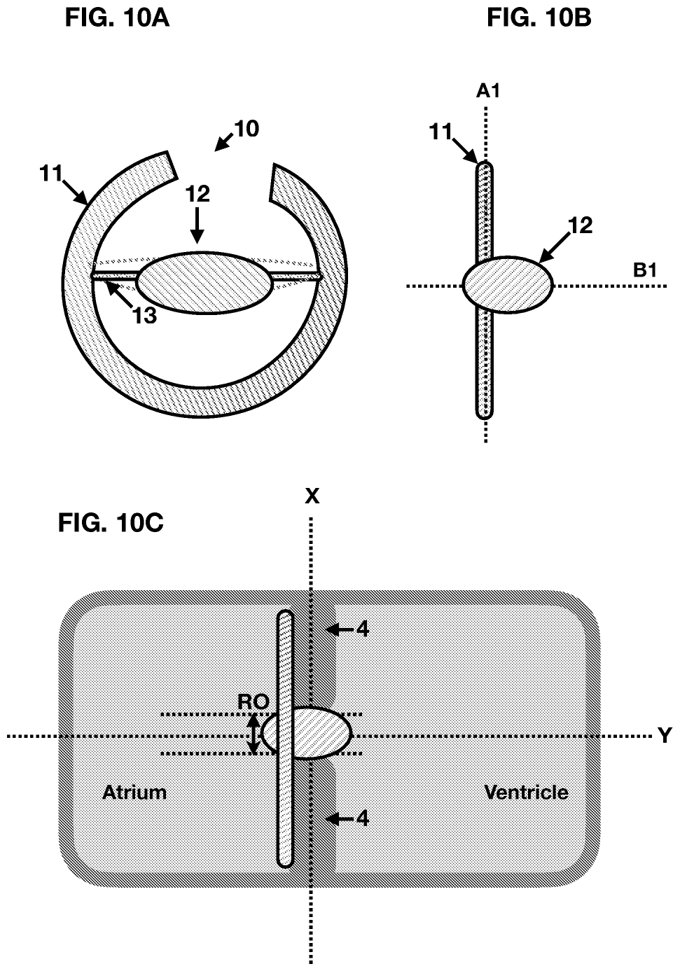

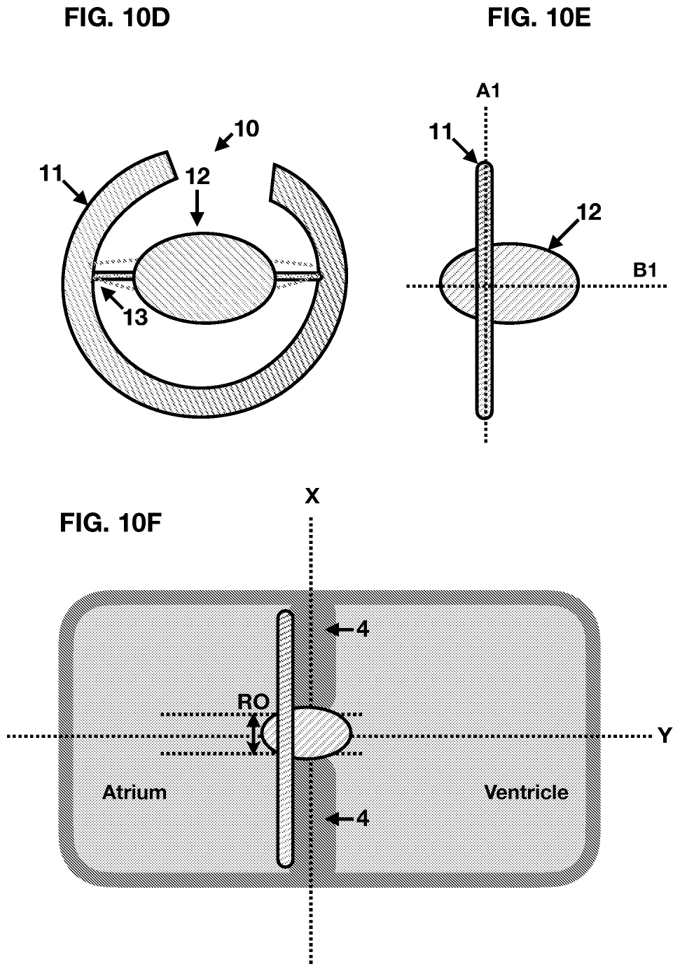

Another embodiment for the mitral valve is illustrated in FIGS. 10A-C. Referring to FIGS. 10A-C, the apparatus 10 comprises a substantially annular support member 11 and a central member 12 securely connected to the annular support member 11 and the connection member 13. As shown in FIG. 10A, for example, the substantially annular support member 11 can be a U-shaped and is dimensioned for attachment to the annulus of the diseased mitral valve 2 as shown in FIG. 1B. As shown in FIG. 10B, the annular support member 11 includes a longitudinal axis A1. The substantially annular support 11 can have a rigid or semi-rigid configuration.

As shown in FIG. 10B, the central member 12 includes a longitudinal axis B1. The central member 12 has a proximal portion and a distal portion 15. As FIG. 10B shows a top view of the apparatus 10 of FIG. 10A, the central member 12 is dimensioned, shaped, and configured to expand horizontally through the RO 3 toward the ventricle when the apparatus 10 is implanted on or about the diseased mitral valve 2 as shown in FIG. 10C.

As shown in FIG. 10C, the central member 12 has a configuration where the central member 12 expands horizontally through the RO 3 (i.e., between the diseased leaflets 4) with an angle between A1 and B1 as shown in FIG. 9B. The angle is such that the central member 12 can induce the diseased leaflets 4 to contact toward to the central member 12. In one example of the embodiment, the angle of A1 and B1 can be about 80 and about 100 (e.g., about 90 degree). To induce the diseased leaflet 4 to contact toward to the central member 12, the dimension of the central member 12 can be selectively adjusted depending upon the anatomic leaflet configuration. It will be appreciated that the adjustment mechanism can be operated to adjust the lateral position of the central member 12 to adjust the angle defined between A1 and B1.

Referring to FIG. 10A-C, the central member 12 can have a rigid or semi-rigid configuration. Where the central member 12 has a semi-rigid configuration, for example, the central member 12 can be bendable or adjustable to various positions. The central member 12 can have a 3D shape that corresponds to the 3D shape of the valve leaflets 4. The engaging portion of the central member 12 has a oval shape. Alternatively, the central member 12 can have a convex shape relative to axis B1. It should be appreciated that the rigidity and shape of the central member 12 can be varied depending upon the diseased mitral valve anatomy for optimal leaflet coaptation. For example, FIGS. 10D-10F show the central member 12 may have a more convex or projecting shape than the one of FIGS. 10A-10C depending on patients.

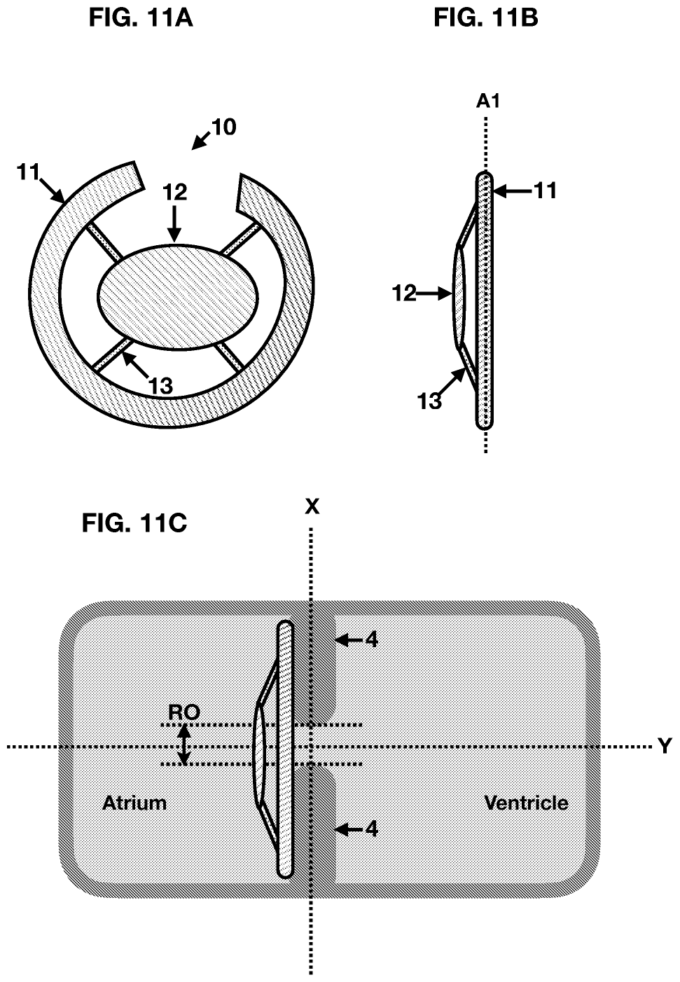

Another embodiment for the mitral valve is illustrated in FIGS. 11A-C. Referring to FIGS. 11A-C, the apparatus 10 comprises a substantially annular support member 11 and a central member 12 securely connected to the annular support member 11 and the connection member 13. As shown in FIG. 11A, for example, the substantially annular support member 11 can be a U-shaped and is dimensioned for attachment to the annulus of the diseased mitral valve 2 as shown in FIG. 1B. As shown in FIG. 11B, the annular support member 11 includes a longitudinal axis A1. The substantially annular support 11 can have a rigid or semi-rigid configuration.

The central member 12 includes a longitudinal axis B1. As FIG. 11B shows a top view of the apparatus 10 of FIG. 11A, the central member 12 is dimensioned, shaped, and configured to position within the atrium when the apparatus 10 is implanted on or about the diseased mitral valve 2 as shown in FIG. 11C. As shown in FIG. 11C, the central member 12 has a configuration where the central member 12 is positioned within the atrium. To induce the diseased leaflet 4 to contact toward to the central member 12, the dimension of the central member 12 can be selectively adjusted depending upon the anatomic leaflet configuration.

Referring to FIG. 11A-C, the central member 12 can have a rigid or semi-rigid configuration. Where the central member 12 has a semi-rigid configuration, for example, the central member 12 can be bendable or adjustable to various positions. The engaging portion of the central member 12 has a oval shape. It should be appreciated that the rigidity and shape of the central member 12 can be varied depending upon the diseased mitral valve anatomy for optimal leaflet coaptation.

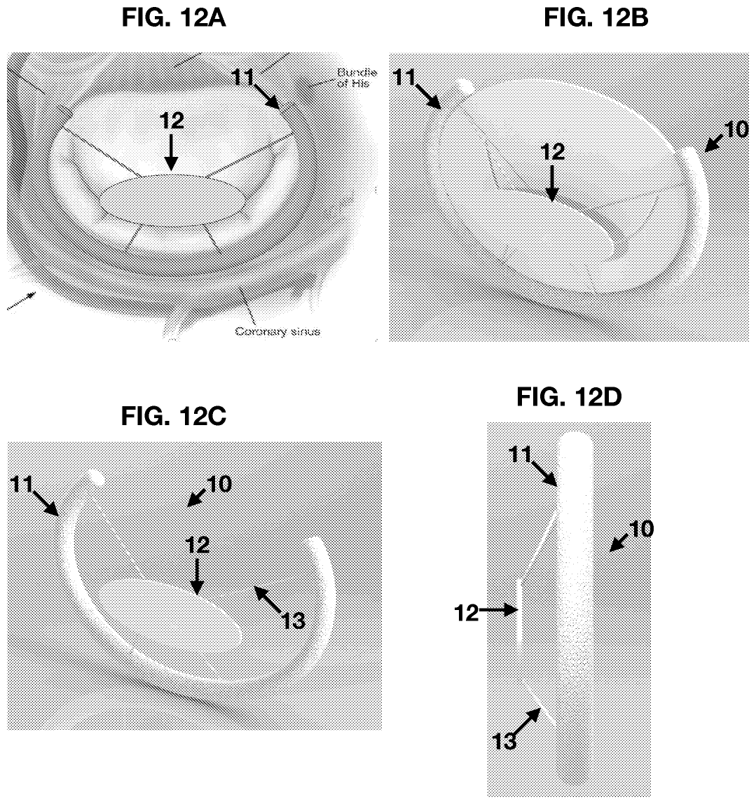

Another embodiment for the mitral valve is illustrated in FIGS. 12A-D. Referring to FIGS. 12A-D, the apparatus 10 comprises a substantially annular support member 11 and a central member 12 securely connected to the annular support member 11 and the connection member 13. As shown in FIG. 11A, for example, the substantially annular support member 11 can be a U-shaped and is dimensioned for attachment to the annulus of the diseased mitral valve 2 as shown in FIG. 1B.

As shown in FIG. 12C, the central member 12 has a configuration where the central member 12 is positioned within the atrium. To induce the diseased leaflet 4 to contact toward to the central member 12, the dimension of the central member 12 can be selectively adjusted depending upon the anatomic leaflet configuration.

Referring to FIG. 12A-D, the central member 12 can have a rigid or semi-rigid configuration. Where the central member 12 has a semi-rigid configuration, for example, the central member 12 can be bendable or adjustable to various positions. The engaging portion of the central member 12 has a oval shape. It should be appreciated that the rigidity and shape of the central member 12 can be varied depending upon the diseased mitral valve anatomy for optimal leaflet coaptation

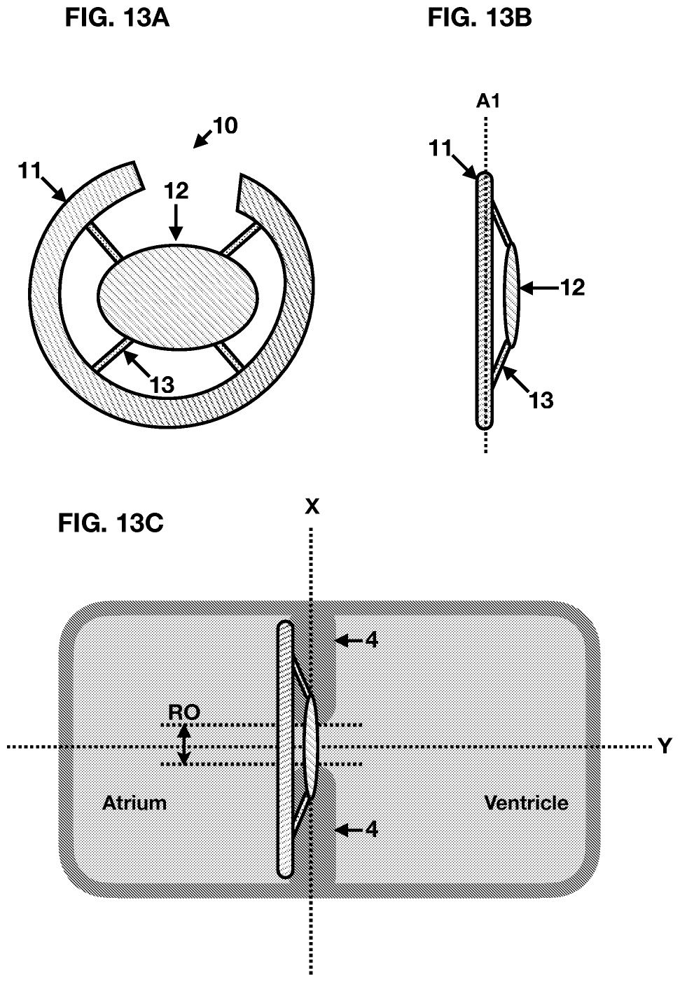

Another embodiment for the mitral valve is illustrated in FIGS. 13A-C.

Referring to FIGS. 13A-C, the apparatus 10 comprises a substantially annular support member 11 and a central member 12 securely connected to the annular support member 11 and the connection member 13. As shown in FIG. 13A, for example, the substantially annular support member 11 can be a U-shaped and is dimensioned for attachment to the annulus of the diseased mitral valve 2 as shown in FIG. 1B. As shown in FIG. 13B, the annular support member 11 includes a longitudinal axis A1. The substantially annular support 11 can have a rigid or semi-rigid configuration.

The central member 12 includes a longitudinal axis B1. As FIG. 13B shows a top view of the apparatus 10 of FIG. 13A, the central member 12 is dimensioned, shaped, and configured to position within the leaflets 4 when the apparatus 10 is implanted on or about the diseased mitral valve 2 as shown in FIG. 13C. As shown in FIG. 13C, the central member 12 has a configuration where the central member 12 is positioned within the leaflets 4. To induce the diseased leaflet 4 to contact toward to the central member 12, the dimension of the central member 12 can be selectively adjusted depending upon the anatomic leaflet configuration.

Referring to FIG. 13A-C, the central member 12 can have a rigid or semi-rigid configuration. Where the central member 12 has a semi-rigid configuration, for example, the central member 12 can be bendable or adjustable to various positions. The central member 12 can have a 3D shape that corresponds to the 3D shape of the valve leaflets 4. The engaging portion of the central member 12 has an oval shape. Alternatively, the central member 12 can have a convex shape relative to axis B1. It should be appreciated that the rigidity and shape of the central member 12 can be varied depending upon the diseased mitral valve anatomy for optimal leaflet coaptation.

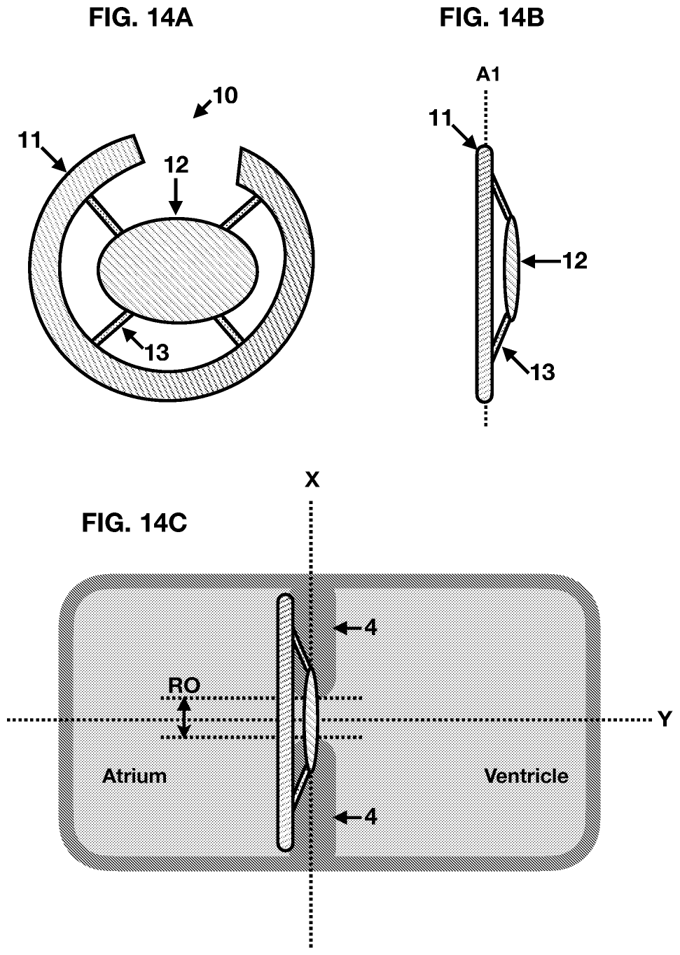

Another embodiment for the mitral valve is illustrated in FIGS. 14A-C. Referring to FIGS. 14A-C, the apparatus 10 comprises a substantially annular support member 11 and a central member 12 securely connected to the annular support member 11 and the connection member 13. As shown in FIG. 14A, for example, the substantially annular support member 11 can be a U-shaped and is dimensioned for attachment to the annulus of the diseased mitral valve 2 as shown in FIG. 1B. As shown in FIG. 14B, the annular support member 11 includes a longitudinal axis A1. The substantially annular support 11 can have a rigid or semi-rigid configuration.

The central member 12 includes a longitudinal axis B1. As FIG. 14B shows a top view of the apparatus 10 of FIG. 14A, the central member 12 is dimensioned, shaped, and configured to position within the diseased mitral valve leaflets 4 when the apparatus 10 is implanted on or about the diseased mitral valve 2 as shown in FIG. 14C. As shown in FIG. 14C, the central member 12 has a configuration where the central member 12 is positioned within the leaflets 4. To induce the diseased leaflet 4 to contact toward to the central member 12, the dimension of the central member 12 can be selectively adjusted depending upon the anatomic leaflet configuration.

Referring to FIG. 14A-C, the central member 12 can have a rigid or semi-rigid configuration. Where the central member 12 has a semi-rigid configuration, for example, the central member 12 can be bendable or adjustable to various positions. The engaging portion of the central member 12 has a oval shape. Alternatively, the central member 12 can have a convex shape relative to axis B1. It should be appreciated that the rigidity and shape of the central member 12 can be varied depending upon the diseased mitral valve anatomy for optimal leaflet coaptation.

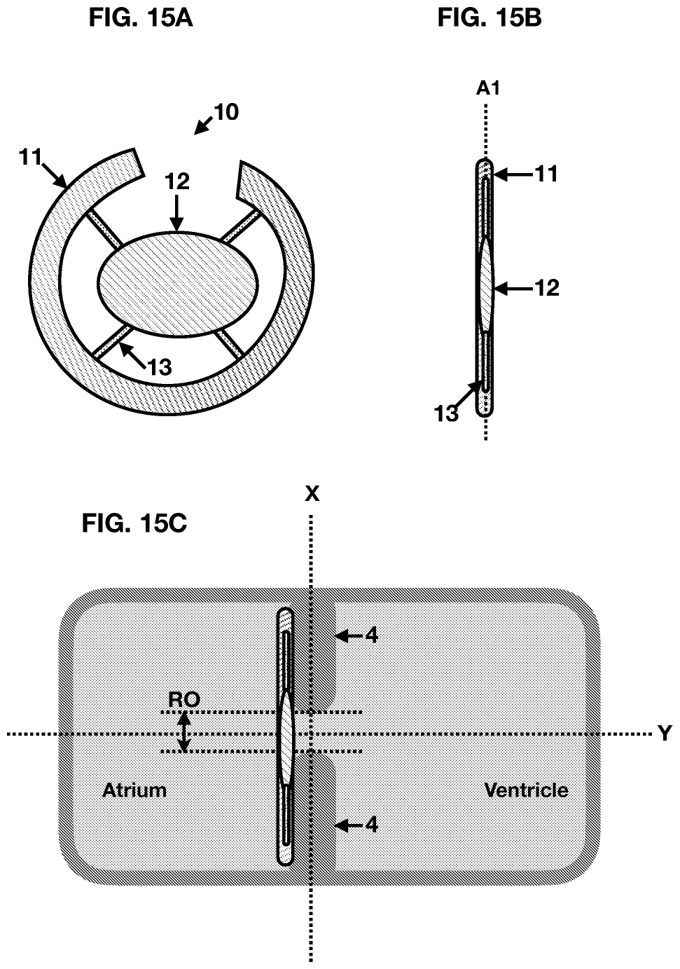

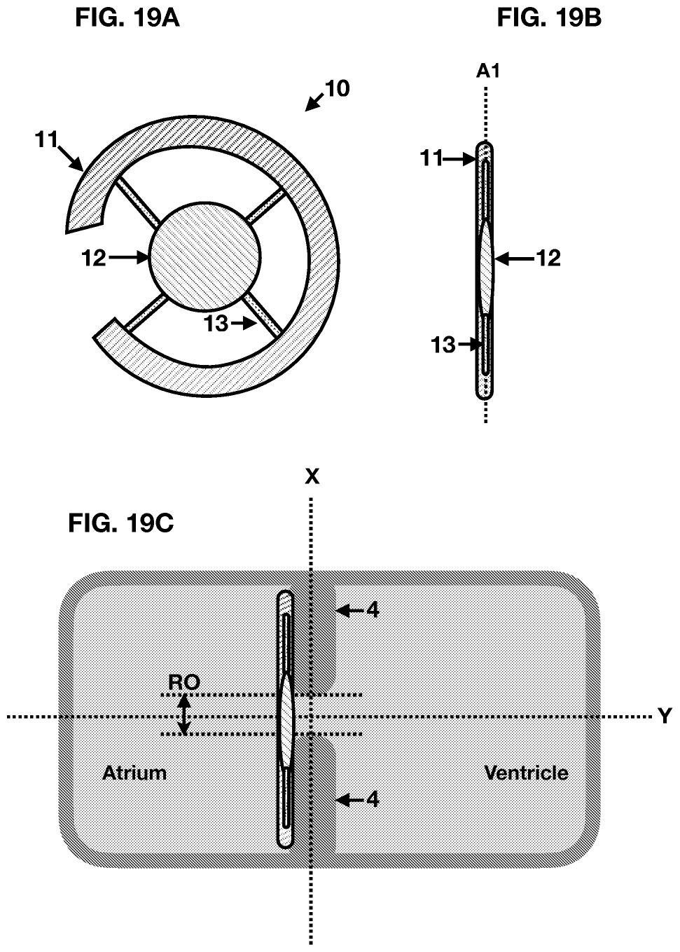

Another embodiment for the mitral valve is illustrated in FIGS. 15A-C. Referring to FIGS. 15A-C, the apparatus 10 comprises a substantially annular support member 11 and a central member 12 securely connected to the annular support member 11 and the connection member 13. As shown in FIG. 19A, for example, the substantially annular support member 11 can be a U-shaped and is dimensioned for attachment to the annulus of the diseased mitral valve 2 as shown in FIG. 1B.

As shown in FIG. 15B, the annular support member 11 includes a longitudinal axis A1. The substantially annular support 11 can have a rigid or semi-rigid configuration. As FIG. 15B shows a top view of the apparatus 10 of FIG. 19A, the central member 12 is dimensioned, shaped, and configured to position proximate the RO3 toward the atrium when the apparatus 10 is implanted on or about the diseased tricuspid valve 1 as shown in FIG. 15C. As shown in FIG. 15C, the central member 12 has a configuration where the central member 12 is positioned proximate the leaflets 4 toward the atrium. To induce the diseased leaflet 4 to contact toward to the central member 12, the dimension of the central member 12 can be selectively adjusted depending upon the anatomic leaflet configuration.

Referring to FIG. 15A-C, the central member 12 can have a rigid or semi-rigid configuration. Where the central member 12 has a semi-rigid configuration, for example, the central member 12 can be bendable or adjustable to various positions. The engaging portion of the central member 12 has a oval shape. It should be appreciated that the rigidity and shape of the central member 12 can be varied depending upon the diseased mitral valve anatomy for optimal leaflet coaptation.

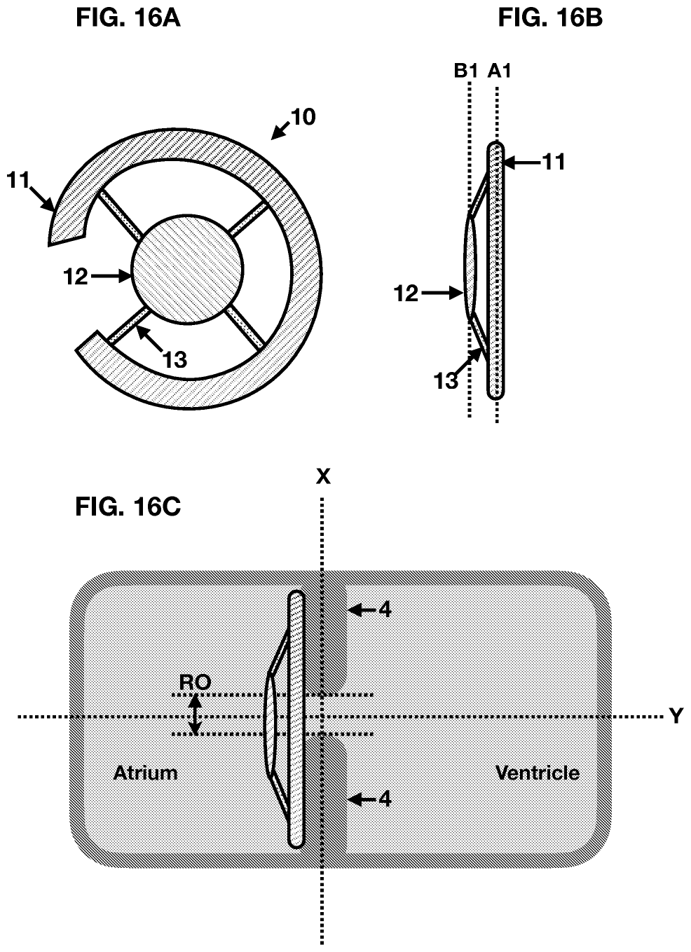

Another embodiment for the tricuspid valve is illustrated in FIGS. 16A-C. Referring to FIGS. 16A-C, the apparatus 10 comprises a substantially annular support member 11 and a central member 12 securely connected to the annular support member 11 and the connection member 13. As shown in FIG. 16A, for example, the substantially annular support member 11 can be a reversed C-shaped and is dimensioned for attachment to the annulus of the diseased tricuspid valve 1 as shown in FIG. 1A.

As shown in FIG. 16B, the annular support member 11 includes a longitudinal axis A1. The substantially annular support 11 can have a rigid or semi-rigid configuration. As FIG. 16B shows a top view of the apparatus 10 of FIG. 16A, the central member 12 is dimensioned, shaped, and configured to position on or upon the leaflets toward to the atrium when the apparatus 10 is implanted on or about the diseased tricuspid valve 1 as shown in FIG. 16C. As shown in FIG. 16C, the central member 12 has a configuration where the central member 12 is positioned behind the annular support member toward to the atrium. To induce the diseased leaflet 4 to contact toward to the central member 12, the dimension of the central member 12 can be selectively adjusted depending upon the anatomic leaflet configuration.

Referring to FIG. 16A-C, the central member 12 can have a rigid or semi-rigid configuration. Where the central member 12 has a semi-rigid configuration, for example, the central member 12 can be bendable or adjustable to various positions. The engaging portion of the central member 12 has a oval shape. Alternatively, the central member 12 can have a convex shape relative to axis B1. It should be appreciated that the rigidity and shape of the central member 12 can be varied depending upon the diseased mitral valve anatomy for optimal leaflet coaptation.

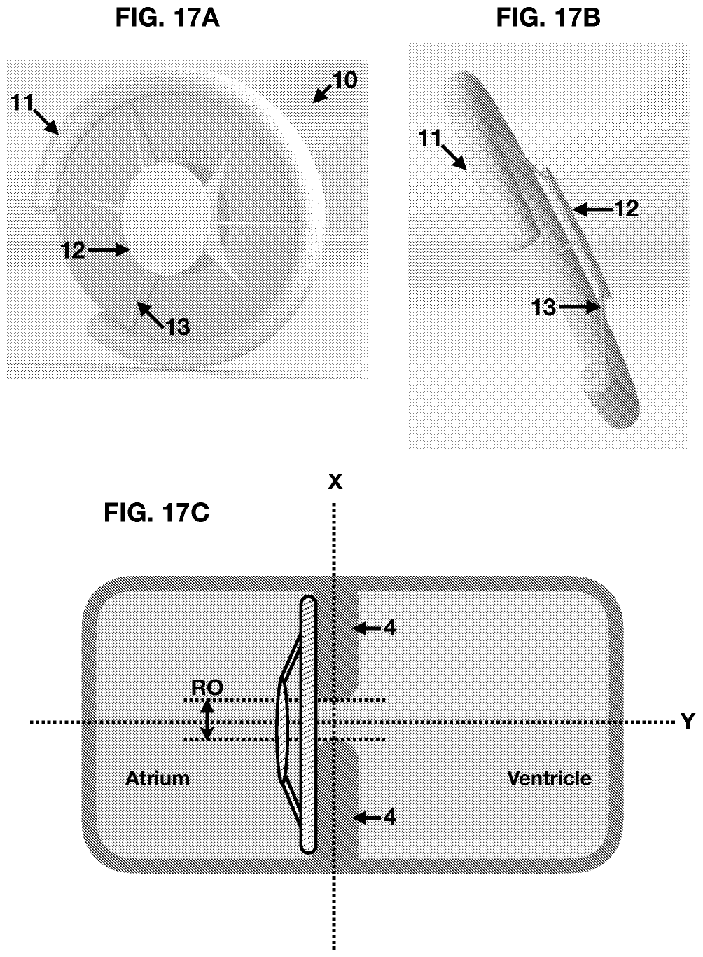

FIGS. 17A-B are perspective views of the apparatus of FIG. 16A. As shown in FIG. 17C, the central member 12 has a configuration where the central member 12 is positioned behind the annular support member toward to the atrium. To induce the diseased leaflet 4 to contact toward to the central member 12, the dimension of the central member 12 can be selectively adjusted depending upon the anatomic leaflet configuration.

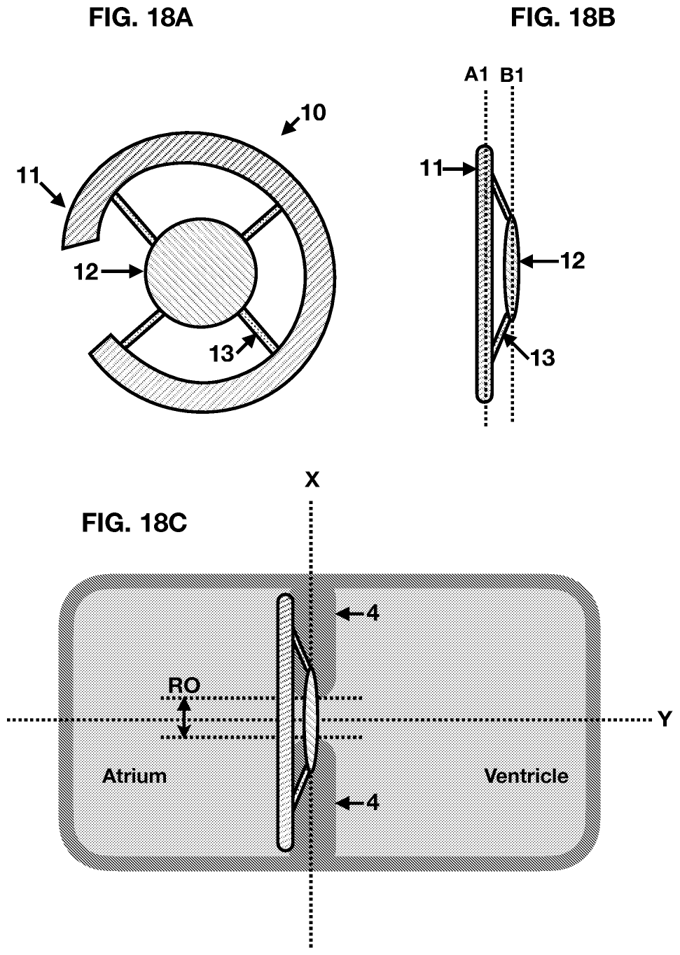

Another embodiment for the tricuspid valve is illustrated in FIGS. 18A-C. Referring to FIGS. 18A-C, the apparatus 10 comprises a substantially annular support member 11 and a central member 12 securely connected to the annular support member 11 and the connection member 13. As shown in FIG. 18A, for example, the substantially annular support member 11 can be a reversed C-shaped and is dimensioned for attachment to the annulus of the diseased tricuspid valve 1 as shown in FIG. 1A.

As shown in FIG. 18B, the annular support member 11 includes a longitudinal axis A1. The substantially annular support 11 can have a rigid or semi-rigid configuration. As FIG. 18B shows a top view of the apparatus 10 of FIG. 18A, the central member 12 is dimensioned, shaped, and configured to position within the leaflets 4 (RO 3) when the apparatus 10 is implanted on or about the diseased tricuspid valve 1 as shown in FIG. 18C. As shown in FIG. 18C, the central member 12 has a configuration where the central member 12 is positioned within the leaflets 4. To induce the diseased leaflet 4 to contact toward to the central member 12, the dimension of the central member 12 can be selectively adjusted depending upon the anatomic leaflet configuration.

Referring to FIG. 18A-C, the central member 12 can have a rigid or semi-rigid configuration. Where the central member 12 has a semi-rigid configuration, for example, the central member 12 can be bendable or adjustable to various positions. The engaging portion of the central member 12 has a oval shape. Alternatively, the central member 12 can have a convex shape relative to axis B1. It should be appreciated that the rigidity and shape of the central member 12 can be varied depending upon the diseased mitral valve anatomy for optimal leaflet coaptation.

Another embodiment for the tricuspid valve is illustrated in FIGS. 19A-C. Referring to FIGS. 19A-C, the apparatus 10 may comprise a substantially annular support member 11 and a central member 12 securely connected to the annular support member 11 and the connection member 13. As shown in FIG. 19A, for example, the substantially annular support member 11 may be a reversed C-shaped and is dimensioned for attachment to the annulus of the diseased tricuspid valve 1 as shown in FIG. 1A.

As shown in FIG. 19B, the annular support member 11 may include a longitudinal axis A1. The substantially annular support 11 may have a rigid or semi-rigid configuration. As FIG. 19B shows a top view of the apparatus 10 of FIG. 19A, the central member 12 is dimensioned, shaped, and configured to position proximate the RO toward the atrium when the apparatus 10 is implanted on or about the diseased tricuspid valve 1 as shown in FIG. 19C. As shown in FIG. 19C, the central member 12 has a configuration where the central member 12 may be positioned proximate the leaflets 4 toward the atrium. To induce the diseased leaflet 4 to contact toward to the central member 12, the dimension of the central member 12 may be selectively adjusted depending upon the anatomic leaflet configuration.

Referring to FIG. 19A-C, the central member 12 can have a rigid or semi-rigid configuration. Where the central member 12 has a semi-rigid configuration, for example, the central member 12 can be bendable or adjustable to various positions. The engaging portion of the central member 12 has an oval shape. It should be appreciated that the rigidity and shape of the central member 12 can be varied depending upon the diseased mitral valve anatomy for optimal leaflet coaptation.

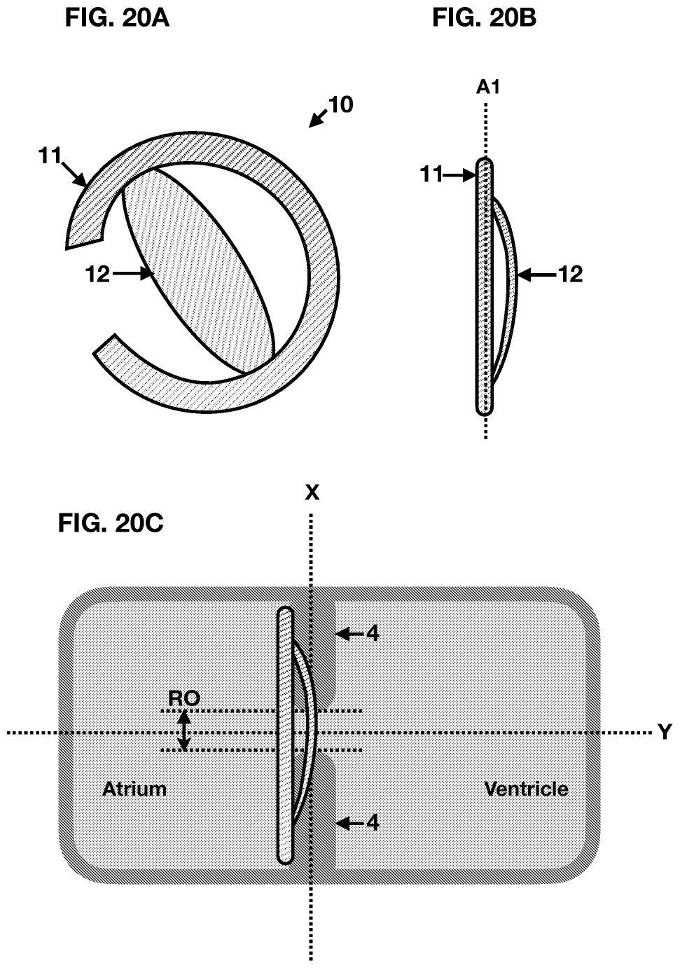

Another embodiment for the tricuspid valve is illustrated in FIGS. 20A-C. Referring to FIGS. 20A-C, the apparatus 10 may comprise a substantially annular support member 11 and a central member 12 securely connected to the annular support member 11. As shown in FIG. 20A, for example, the substantially annular support member 11 may be a reversed C-shaped and is dimensioned for attachment to the annulus of the diseased tricuspid valve 1 as shown in FIG. 1A.

As shown in FIG. 20B, the annular support member 11 may include a longitudinal axis A1. The substantially annular support 11 can have a rigid or semi-rigid configuration. As FIG. 20B shows a top view of the apparatus 10 of FIG. 20A, the central member 12 may be dimensioned, shaped, and configured to position within the leaflets 4 when the apparatus 10 is implanted on or about the diseased tricuspid valve 1 as shown in FIG. 20C. As shown in FIG. 20C, the central member 12 has a configuration where the central member 12 may be positioned within the leaflets 4. To induce the diseased leaflet 4 to contact toward to the central member 12, the dimension of the central member 12 can be selectively adjusted depending upon the anatomic leaflet configuration.

Referring to FIG. 20A-C, the central member 12 can have a rigid or semi-rigid configuration. Where the central member 12 has a semi-rigid configuration, for example, the central member 12 can be bendable or adjustable to various positions. The engaging portion of the central member 12 has an oval shape. It should be appreciated that the rigidity and shape of the central member 12 can be varied depending upon the diseased mitral valve anatomy for optimal leaflet coaptation.

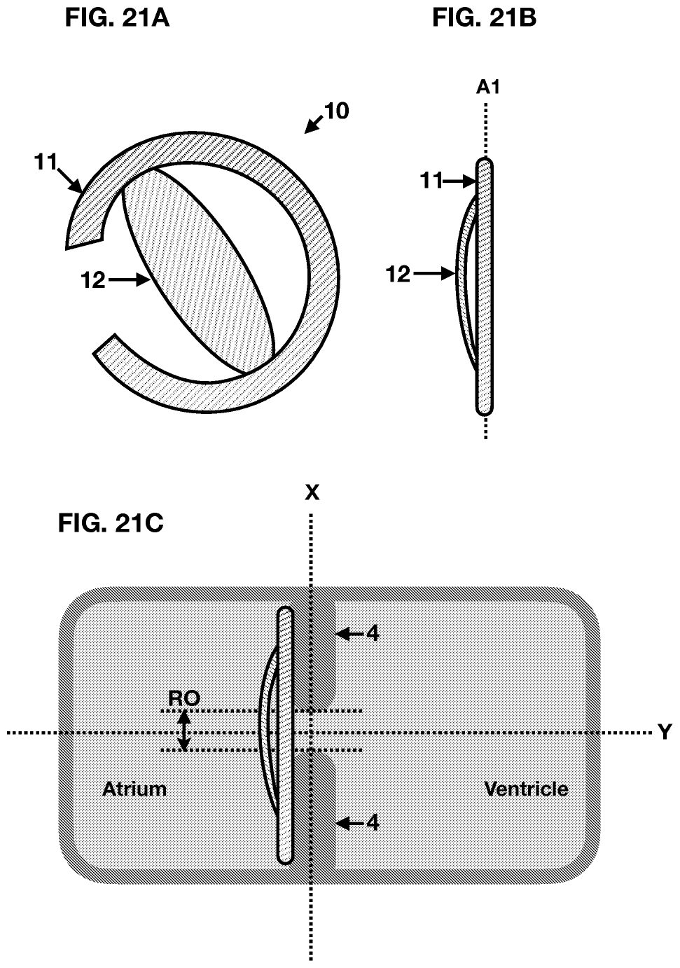

Another embodiment for the tricuspid valve is illustrated in FIGS. 21A-C. Referring to FIGS. 21A-C, the apparatus 10 may comprise a substantially annular support member 11 and a central member 12 securely connected to the annular support member 11. As shown in FIG. 21A, for example, the substantially annular support member 11 can be a reversed C-shaped and is dimensioned for attachment to the annulus of the diseased tricuspid valve 1 as shown in FIG. 1A.

As shown in FIG. 21B, the annular support member 11 may include a longitudinal axis A1. The substantially annular support 11 may have a rigid or semi-rigid configuration. As FIG. 21B shows a top view of the apparatus 10 of FIG. 21A, the central member 12 is dimensioned, shaped, and configured to position proximate the RO toward the atrium when the apparatus 10 is implanted on or about the diseased tricuspid valve 1 as shown in FIG. 21C. As shown in FIG. 21C, the central member 12 may have a configuration where the central member 12 is positioned proximate the leaflets 4 toward the atrium. To induce the diseased leaflet 4 to contact toward to the central member 12, the dimension of the central member 12 can be selectively adjusted depending upon the anatomic leaflet configuration. Referring to FIG. 21A-C, the central member 12 may have a rigid or semi-rigid configuration. Where the central member 12 has a semi-rigid configuration, for example, the central member 12 can be bendable or adjustable to various positions. The engaging portion of the central member 12 has a oval shape. It should be appreciated that the rigidity and shape of the central member 12 can be varied depending upon the diseased mitral valve anatomy for optimal leaflet coaptation.

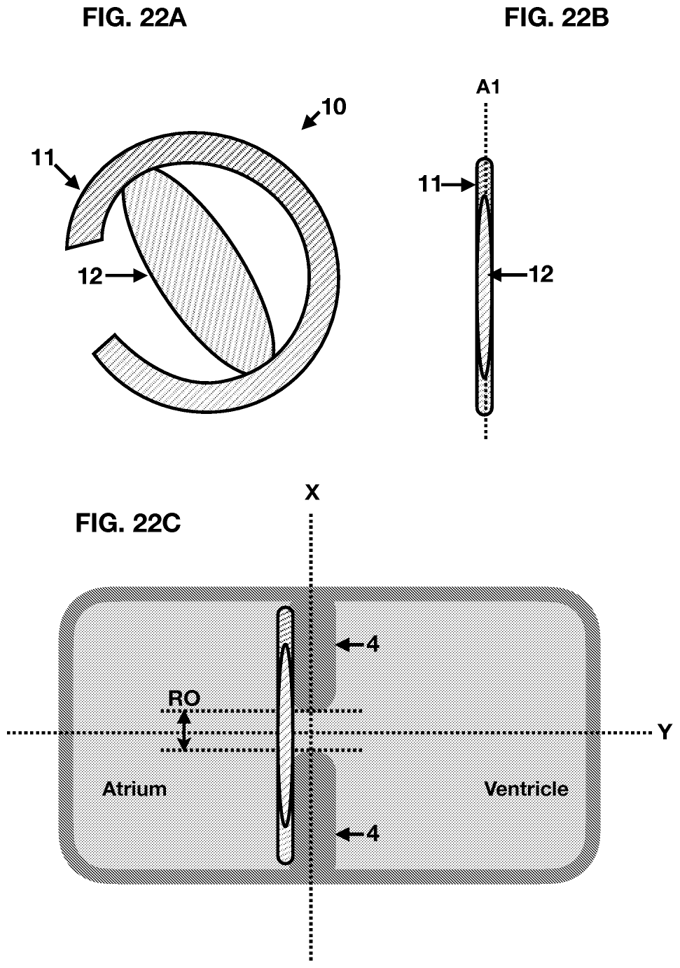

Another embodiment for the tricuspid valve is illustrated in FIGS. 22A-C. Referring to FIGS. 22A-C, the apparatus 10 may comprise a substantially annular support member 11 and a central member 12 securely connected to the annular support member 11. As shown in FIG. 22A, for example, the substantially annular support member 11 may be a reversed C-shaped and may be dimensioned for attachment to the annulus of the diseased tricuspid valve 1 as shown in FIG. 1A.

As shown in FIG. 22B, the annular support member 11 may include a longitudinal axis A1. The substantially annular support 11 can have a rigid or semi-rigid configuration. As FIG. 22B shows a top view of the apparatus 10 of FIG. 22A, the central member 12 is dimensioned, shaped, and configured to position proximate the RO toward the atrium and within the same plane as the annular support member when the apparatus 10 is implanted on or about the diseased tricuspid valve 1 as shown in FIG. 22C. As shown in FIG. 22C, the central member 12 has a configuration where the central member 12 is positioned proximate the leaflets 4 toward the atrium. To induce the diseased leaflet 4 to contact toward to the central member 12, the dimension of the central member 12 can be selectively adjusted depending upon the anatomic leaflet configuration.

Referring to FIG. 22A-C, the central member 12 may have a rigid or semi-rigid configuration. Where the central member 12 has a semi-rigid configuration, for example, the central member 12 can be bendable or adjustable to various positions. The engaging portion of the central member 12 has an oval shape. It should be appreciated that the rigidity and shape of the central member 12 can be varied depending upon the diseased mitral valve anatomy for optimal leaflet coaptation.

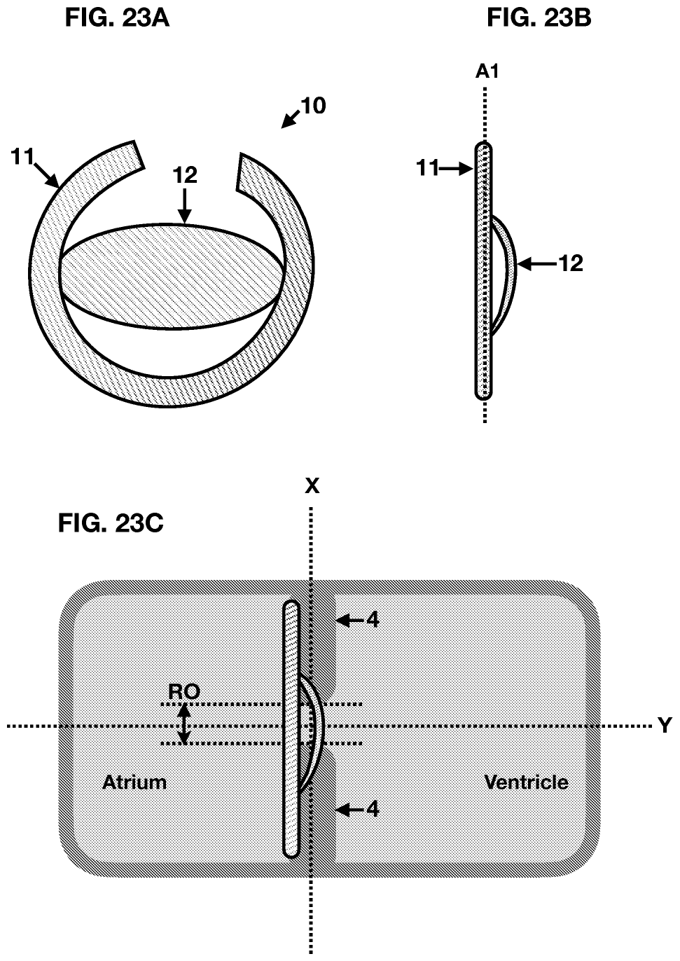

Another embodiment for the mitral valve is illustrated in FIGS. 23A-C. Referring to FIGS. 23A-C, the apparatus 10 may comprise a substantially annular support member 11 and a central member 12 securely connected to the annular support member 11. As shown in FIG. 23A, for example, the substantially annular support member 11 may be a U-shaped and may be dimensioned for attachment to the annulus of the diseased mitral valve 2 as shown in FIG. 1B.

As shown in FIG. 23B, the annular support member 11 may include a longitudinal axis A1. The substantially annular support 11 may have a rigid or semi-rigid configuration. As FIG. 23B shows a top view of the apparatus 10 of FIG. 23A, the central member 12 may be dimensioned, shaped, and configured to position within the leaflets 4 when the apparatus 10 may be implanted on or about the diseased mitral valve 1 as shown in FIG. 22C. As shown in FIG. 23C, the central member 12 may have a configuration where the central member 12 may be positioned within the leaflets 4. To induce the diseased leaflet 4 to contact toward to the central member 12, the dimension of the central member 12 may be selectively adjusted depending upon the anatomic leaflet configuration.

Referring to FIG. 23A-C, the central member 12 may have a rigid or semi-rigid configuration. Where the central member 12 has a semi-rigid configuration, for example, the central member 12 may be bendable or adjustable to various positions. The engaging portion of the central member 12 has an oval shape. It should be appreciated that the rigidity and shape of the central member 12 can be varied depending upon the diseased mitral valve anatomy for optimal leaflet coaptation.

The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

1. An implantable device for repairing a cardiac valve having an annulus, two or more leaflets, the device comprising:
an annual member having a longitudinal axis, the annual member being dimensioned to attach to the annulus of the cardiac valve; and
a central member having a longitudinal axis, a distal portion, and a proximal portion, the central member obliquely attached to the annual member in such a way the distal portion of the central member obliquely traverses the leaflets toward a ventricle to induce the leaflets to contact toward to the central member.

2. The device according to claim 1, the proximal portion of the central member is configured to be placed in an atrium.

3. The device according to claim 1, further comprising at least one connection member configured to support the central member from the annular member.

4. The device according to claim 1, the central member has a shape of a croissant.

5. An implantable device for repairing a cardiac valve having an annulus, two or more leaflets, the device comprising:
an annual member having a longitudinal axis, the annual member being dimensioned to attach to the annulus of the cardiac valve; and
a central member having a longitudinal axis, a distal portion, and a proximal portion, the central member obliquely attached to the annual member in such a way the longitudinal axes of the annual member and the central member have a predetermined angle to induce the leaflets to contact toward to the central member.

6. The device according to claim 5, the distal portion of the central member is configured to traverse the leaflets of the cardiac valve into a ventricle while the proximal portion of the central member is placed in an atrium.

7. The device according to claim 5, the entire central member is configured to be placed in between the leaflets.

8. The device according to claim 5, the entire central member is configured to be placed in an atrium.

9. The device according to claim 5, the entire central member is configured to be placed in a ventricle.

10. The device according to claim 5, the predetermined angle is about zero degree.

11. The device according to claim 5, the predetermined angle is about 90 degree.

12. The device according to claim 5, further comprising at least one connection member configured to support the central member from the annular member.

13. The device according to claim 5, the central member has a shape of a croissant.
